(19) United States
(12) Patent Application Publication
(10) Pub. No.: US 2011/0229411 A1
(43) Pub. Date: Sep. 22, 2011

(54) USE OF P2X7 PATHWAY FOR ASSESSING THE SENSITIVITY OF A SUBJECT TO A CANCER TREATMENT

(75) Inventors: Lionel Apetoh, Boston, MA (US); Antoine Tesniere, Paris (FR); François Ghiringhelli, Dijon (FR); Laurence Zitvogel, Antony (FR); Guido Kroemer, Antony (FR)

(73) Assignees: INSTITUT GUSTAVE ROUSSY, VILLEJUIF (FR); INSTITUT NATIONAL DE LA SANTÉ ET DE LA RECHERCHE MÉDICALE (INSERM), PARIS CEDEX 13 (FR); UNIVERSITE PARIS-SUD XI, ORSAY CEDEX (FR)

(21) Appl. No.: 13/131,154

(22) PCT Filed: Nov. 27, 2008
(86) PCT No.: PCT/IB2008/055658

§ 371 Date: May 25, 2011

(51) Int. Cl. A61K 49/00; C12Q 1/02; A61P 35/00; A61K 38/20; A61K 38/02; A61K 38/12; A61K 39/395; A61K 38/21; C12Q 1/68; C12Q 1/37

(52) U.S. Cl. 424/9.1; 435/29; 424/85.2; 514/19.3; 514/19.9; 424/172.1; 424/145.1; 424/85.7; 435/6.11; 435/23

(57) ABSTRACT

The present invention concerns methods for assessing the sensibility of a subject to an anticancer treatment, for screening compounds which are useful for treating a cancer and for determining the likelihood of a metastatic relapse in a subject. The methods are based on the finding that a non-functional P2X7-elicited NALP3 inflammasome pathway in a subject is indicative of a resistance to treatment. The invention further concerns methods for treating a cancer and for restoring the sensitivity of the subject to a cancer treatment.

### FIELD OF THE INVENTION

The present disclosure generally relates to the fields of genetics, immunology and medicine. The inventors more particularly disclose the identification of a pathway used to predict or assess the sensitivity of a subject to a treatment of cancer. This pathway can also be used for the screening of therapeutically active drugs and to restore the sensitivity.

### BACKGROUND OF THE INVENTION

Cancer occurs when cell division gets out of control and results from impairment of a DNA repair pathway, the transformation of a normal gene into an oncogene or the malfunction of a tumor supressor gene.

Along with surgery, chemotherapy and radiotherapy are used for treating many types of cancer. A large variety of chemotherapeutic agents, involving various mechanisms, has been developed and the survival of cancer patients has been greatly improved for many type of cancers. However, one of the most important problems in cancer treatment remains that individual subjects with the same histology do not respond identically to a given agent or a given therapeutic protocol.

Until recently, the role of the immune system in cancer diseases was only considered for his pro-tumoral aspects. However, radiotherapy and some chemotherapeutic agents could also induce specific immune responses resulting in immunogenic cancer cell death or in immunostimulatory side effects (Koebel et al., 2007; Zitvogel et al., 2008). The impact of anticancer therapies on immune responses can involve distinct mechanisms (i) the elimination of immunosuppressive cells such as regulatory T cells (Tregs) (e.g. cyclophosphamide) or myeloid suppressor cells (e.g. gemcitabine, ATRA), (ii) the activation of immune effectors (e.g. imatinib mesylate, histone deacetylase inhibitors), and (iii) induction of an immunogenic cancer cell death (e.g. anthracyclines, oxaliplatin and X-rays).

When the treatment involves agents inducing an immunogenic cancer cell death, efficient response depends on the capacity of dendritic cells (DC) to present antigen from dying cancer cells and to prime tumor-specific cytotoxic T lymphocytes (CTL). To mount a CTL response, DC must incorporate antigens from stressed or dying cells, acquire the competence of antigen processing in a maturation step and present antigenic peptides bound to MHC molecules in the context of costimulatory signals and cytokines that stimulate the differentiation/activation of specific CTL (Figdor et al., 2004; Steinman et al., 2007). The phagocytosis of treated tumor cells by dendritic cells is facilitated by pre-apoptotic translocation of calreticulin to the tumor cell surface (Obeid et al., 2007). The activation of tumor antigen-specific T-cell immunity involves secretion of the high-mobility-group box 1 alarmin protein (HMGB1) by dying tumor cells and the action of this protein on Toll-like receptor 4 (TLR4) expressed by dendritic cells (Apetoh et al., 2007). Based on these observations, an abnormal TLR4 protein expression or activity has been previously described as being indicative of a resistance to an anticancer treatment (WO 08/009693).

Since anthracyclines, oxaliplatin and X-Rays represent the basis of the majority of anticancer treatments, prediction of reduced response to these treatments are essential for the patient management. Moreover, considering that most of anticancer treatments not only cause severe side effects but also are generally physically exhausting for patients and often associated with high costs, the choice of the appropriate chemotherapy and/or radiotherapy protocols is of capital importance. Consequently, there is a strong need for a method for predicting the response of a patient to a particular treatment prior to the actual onset of said treatment. Based on this prediction, the therapeutic protocol may then be adapted for this patient.

### SUMMARY OF THE INVENTION

The P2X₇-elicited NALP3 inflammasome pathway has been surprisingly found by the inventors to be involved in the response of a subject to an anticancer treatment and thus in the sensitivity of said subject to such treatment.

Accordingly, in a first aspect, the present invention concerns an in vitro method of assessing the sensitivity of a subject to a chemotherapeutic or radiotherapeutic treatment of cancer, wherein the method comprises determining the functional status of the P2X₇-elicited NALP3 inflammasome pathway, a non-functional pathway being indicative of a resistance to said treatment.

In a second aspect, the present invention concerns an in vitro method for screening a compound useful for increasing or restoring the sensitivity to a chemotherapeutic or radiotherapeutic treatment of cancer in a subject having a loss of function in the P2X₇-elicited NALP3 inflammasome pathway, wherein the method comprises determining the ability of a test compound to induce or increase IL-10 secretion by dendritic cells in presence of dying tumor cells in said subject.

In a further aspect, the present invention concerns an in vitro method for screening a compound useful for treating a cancer in a subject having a loss of function in the P2X₇-elicited NALP3 inflammasome pathway, wherein the method comprises determining the ability of a test compound to induce or increase IL-1β secretion by dendritic cells in presence of dying tumor cells in said subject.

In another aspect, the present invention concerns a method for screening a compound useful for increasing or restoring the sensitivity to a chemotherapeutic or radiotherapeutic treatment of cancer in a subject having a non-functional P2X₇-elicited NALP3 inflammasome pathway, wherein the method comprises (i) administering a test compound in combination with a chemotherapeutic or radiotherapeutic treatment of cancer to a non-human transgenic animal with non-functional P2X₇-elicited NALP3 inflammasome pathway and inoculated with a tumor, and (ii) assessing the sensitivity of said animal to said treatment.

In another aspect, the present invention concerns an in vitro method for determining the likelihood of a metastatic relapse in a subject, which method comprises determining the functional status of the P2X₇-elicited NALP3 inflammasome pathway in said subject, a non functional pathway being indicative of an increase likelihood of a metastatic relapse.

In another aspect, the present invention concerns an in vitro method for selecting the proper chemotherapeutic or radiotherapeutic treatment for a subject in need thereof, wherein the method comprises determining the functional status of the P2X₇-elicited NALP3 inflammasome pathway, a non functional pathway being considered as a contraindication for an anticancer treatment inducing immunogenic tumor cell death.

In further aspect, the present invention concerns a pharmaceutical composition comprising a chemotherapeutic agent and a compound which is able to compensate a loss of function in the P2X₇-elicited NALP3 inflammasome pathway.

In another aspect, the present invention concerns a product containing a chemotherapeutic agent and a compound which is able to compensate a loss of function in the P2X7-elicited NALP3 inflammasome pathway, as a combined preparation for simultaneous, separate or sequential use in the treatment of cancer.

In another aspect, the present invention concerns a compound which is able to compensate a loss of function in the P2X₇-elicited NALP3 inflammasome pathway, for use in the treatment of a cancer, in combination with a chemotherapeutic agent or a radiotherapeutic treatment, in a subject having a loss of function in the P2X₇-elicited NALP3 inflammasome pathway.

In another aspect, the present invention concerns the use of a compound which is able to compensate a loss of function in the P2X₇-elicited NALP3 inflammasome pathway, for the manufacture of a medicament for treating cancer in a subject having a loss of function in the P2X₇-elicited NALP3 inflammasome pathway.

The present invention also concerns a method for increasing the efficacy of a chemotherapeutic or radiotherapeutic treatment in a subject suffering from a cancer and having a non-functional P2X₇-elicited NALP3 inflammasome pathway, wherein the method comprises administering a chemotherapeutic or radiotherapeutic treatment in combination with a therapeutically effective amount of compound which is able to compensate a loss of function in the P2X₇-elicited NALP3 inflammasome pathway.

### BRIEF DESCRIPTION OF THE DRAWINGS

FIG. 1: ATP release by dying tumor cells dictates the immunogenicity of cell death. FIG. 1a. Accumulation of ATP in the extracellular space following exposure of tumor cells to chemotherapy. EG7 were treated with oxaliplatin, and ATP release into the supernatant was monitored. FIG. 1b. Decrease of intracellular ATP upon oxaliplatin treatment and depletion of the intracellular pool of ATP using antimycin A and deoxyglucose. EG7 were treated with oxaliplatin for 24 hours and subsequently with antimycin A and deoxyglucose (A/D) for 20 min. Data (means±SEM of triplicates) of one representative experiment out of three are depicted. FIG. 1c. Failure of dying tumor cells depleted from ATP to elicit an OVA specific immune response. Live or oxaliplatin-treated EG7 cells were injected into the footpad of C57B1/6 mice. Five days later, popliteal lymph node cells were recovered and restimulated with the OVA holoprotein for 72 hours before quantification of IFNγ secretion. The levels of IFNγ secretion between mice immunized with ATP-depleted or not depleted EG7 were compared in three independent experiments. FIG. 1d. Inhibition of purinergic receptors with oxidized ATP (oxiATP) abolishes T cell priming by oxaliplatin-treated EG7 cells. Oxaliplatin-treated EG7 cells admixed or not with oxiATP were injected into the footpad of C57B1/6 mice and the OVA-specific immune response was examined as in c. and compared with that achieved with OVA protein injected along with TLR3/9 ligands (PolyI:C+CpG (C/P)) in the presence or absence of oxiATP. FIG. 1e. Requirement of P2X7 expression on DC to mediate immune responses against dying tumor cells. DC from WT or P2X7−/− mice were first loaded with antigen (OVA holoprotein with C/P adjuvant, live or oxaliplatin-treated EG7 cells) and then injected into the footpad of P2X7−/− recipients. Five days later, the local immune response was measured as in (a). The experiments included 3-4 mice per group and were repeated three times with similar results. * p<0.05. FIG. 1f. Phenotype of oxaliplatin-treated E G7 prior to or after ATP depletion. Flow cytometry analyses of EG7 tumor cells that were treated for 20 hrs with oxaliplatin, washed and incubated 20 min with antimycin A/deoxyglucose (A/D) (as in FIG. 1b) and stained with propidium iodine and annexin V-FITC.

FIG. 2: Phenotype of oxaliplatin-treated E G7 pr for to or after ATP depletion. Flow cytometry analyses of EG7 tumor cells that were treated for 20 hrs with oxaliplatin, washed and incubated 20 min with antimycin A/deoxyglucose (A/D) (as in FIG. 1b) and stained with propidium iodine and annexin V-FITC.

FIG. 3: Identification of AT P release as a limiting factor of anti-cancer vaccination by dying CT 26 cells. FIG. 3a-b Idem as in FIGS. 1a and b, but using the mouse colon cancer CT26 exposed to doxorubicin. Data of one representative experiment out of three are depicted. FIG. 3. c-d. Depletion of ATP (c) or blockade of purinergic receptors (d) prevent the efficacy of vaccination with dying tumor cells. Mice were immunized with PBS or dying CT26 tumor cells. Dying CT26 were first treated with doxorubicin for 24 hours and were then incubated with antimycin A/deoxyglucose (A/D) for 20 min. Cells were washed after A/D exposure and before subcutaneous inoculation (c). Alternatively, dying CT26 were inoculated alone or together with oxiATP (d). Seven days later, mice (15 per group in a total of 3 distinct experiments) were inoculated with live syngeneic tumor cells and tumor growth was monitored. * p<0.05.

FIG. 4: Purinergic P2X7 receptors are mandatory for the immunogenicity of cell death. Failure of dying tumor cells to elicit an OVA -specific immune response in P2X₇^{-/-} mice. Live or oxaliplatin-treated EG7 cells were injected into the footpad of C57B1/6 mice (WT or P2X₇^{-/-}). Five days later, popliteal lymph node cells were recovered and restimulated with the OV A holoprotein for 72 hours before quantification of IFNγsecretion. As a positive control of antigen presentation, mice were injected with 1 mg of OV A protein plus 10 μg CpG 28 and 5 μg Poly I:C (C/P) as adjuvant.

FIG. 5: Essential contribution of the Nalp3 inflammasome-dependent IL-1β production to cross-priming of T cells for IFNγ production by dying tumor cells.

FIG. 5a-b. Immunofluorescence staining of active caspase-1 in bone marrow-derived DC. DC (WT or derived from P2X₇^{-/-}, Nalp3^{-/-} or Asc^{-/-} mice) were exposed oxaliplatin or to live or oxaliplatin-treated EG7 tumor cells in the presence or absence of oxidized ATP and labeled with anti-CD11c, anti-caspase 1 p20 or isotype control antibodies (not shown) and DAPI (a, b) or left in culture for 24 hours (c). Positive controls included DC exposed to LPS (50 ng/ml) for 16 hrs and then to ATP (2 mM) for 20 min. Representative microphotographs or histograms are depicted in (a). The quantification of DC containing active caspase-1 is depicted in (b).

FIG. 5c. IL-1β secretion by DC recognizing dying tumor cells is dependent on the NALP3 inflammasome. IL-1β levels were measured by commercial ELISA in the supernatants of DC. n.d.: not determined. The experiments were performed three times with similar results. Representative microphotographs or histograms are depicted. Means±SEM are shown for triplicate wells.

FIG. 5d. Failure of dying tumor cells to elicit an OVA-specific immune response in NALP3^{-/-}, Casp1^{-/-} IL-1R1^{-/-} mice. Live or oxaliplatin-treated EG7 cells were injected into the footpad of C57B1/6 mice (WT, Nalp3^{-/-}, Casp1^{-/-} or IL-1R1^{-/-}). Five days later, poplitcal lymph node cells were recovered and restimulated with the OVA holoprotein for 72 hours before quantification of IFNγ secretion. As a positive control of antigen presentation, mice were injected with 1 mg of OVA protein plus C/P adjuvants.

FIG. 5e. Cross-priming of CD8⁺ T cells by dying H-2^{d+}TS/A in H-2^{b-} mice. X Ray-treated breast cancer cells TS/A were injected in C57BL/6 mice bearing a WT or Casp-1^{-/-} genotype (n=3 mice/group). Draining lymph node cells were harvested at day 5, restimulated ex vivo with SIINFEKL peptides or OVA holoprotein for 72 hrs and IFNγ secretion was measured by ELISA in the supernatants. * p<0.05.

FIG. 6: Maturation of DC in response to dying tumor cells is independent of caspase-1. Cytofluorometric analyses of BM-DC (derived from WT or casp-1 or Nalp3 deficient hosts) loaded for 24 h with oxaliplatin-treated tumor cells at a 1:1 ratio. Phenotypic characterization was achieved using three-color staining (using anti-CD11c, I-Ab, and CD80 or CD86 or CD40 mAb). Columns represent the percentage of I-Ab+, CD80+, CD86+ or CD40+ cells among the CD11c+ population. These experiments were performed twice with similar results. * p<0.05.

FIG. 7: IL-12p40 secretion by DC recognizing dying tumor cells is independent on the NAL P3 inflammasome. IL -12p40 levels were measured by commercial ELISA in the same supernatants of DC as those described in FIG. 5c. n.d.: not determined. The experiments were performed three times with similar results. Representative microphotographs or histograms are depicted. Means±SEM are shown for triplicate wells.

FIG. 8: Antigen processing of tumor cell associated antigens by DC is Casp-1 independent. BM-DC derived from WT, Casp-1 and Nalp3 deficient mice were incubated with oxaliplatin-treated EG7 cells (at a 1:1 ratio) and exposed to the B3Z hybridoma (at a 1:2 DC/T cell ratio). Controls included live EG7 cells and SIINFEKL peptide. IL-2 levels were measured with a commercial ELISA at 48 hrs. The experiment has been performed twice with comparable results. The graph depicts means±SEM of triplicates. * p<0.05.

FIG. 9: DC must express NAL P3 and Casp1 to mediate the immunogenicity of oxaliplatin-treated tumor cells. DC from WT, Nalp3−/− or Casp1−/− mice were first loaded with antigen (recombinant OVA protein with a mixture of CpG ODN(10 μg/ml) and poly-I:C (5 μm/ml) (C/P) as adjuvants, live or oxaliplatin-treated EG7 cells and then injected into the footpad of WT recipients. Five days later, the local immune response was measured as IFNγ secretion from in vitro restimulated lymph node T cells, as described in FIG. 5d.

FIG. 10: Effect of the caspase-1 knockout on the production of distinct cytokines by T cells primed in vivo with dying tumor cells. WT or casp1−/− mice were injected into the footpad with oxaliplatin-treated EG7 cells, and five days later draining lymph node cells were restimulated with OVA protein to assess the in vitro production (after 48 h) of IFNγ, IL-4, IL-10, IL-13 and IL-17. While IL-4, IL-17 were undetectable in all cases (not shown), IL-10 and IL-13 were produced at similar levels on all indicated genetic backgrounds. Thus, the absence of caspase-1 selectively impairs the production of IFNγ, not that of IL-10 or IL-13. One representative experiment (mean±SEM of triplicates) out of two is shown. * p<0.05.

FIG. 11: Cross-priming of T cells to cell associated antigens presented by DC loaded with dying cells depends on CASP-1. FIG. 11a. Mouse embryonic fibroblasts (MEF) transfected with cDNA encoding the OVA fused to the transmembrane domain of K^{b} or EG7 were treated with oxaliplatin (5 μg/ml, 24 h) prior to injection into the footpad of WT or Casp-1−/− mice. Popliteal lymph node cells were obtained five days later and were restimulated with MEF, EL 4 lysates, or ovalbumin. IFNγ levels were monitored at 48 hrs by ELISA. FIG. 11b. Dying MEF can induce IL-1β production by DC in vitro. Similar methods as in FIG. 5c. FIG. 11c. OVA-loaded macrophages treated with oxaliplatin induce casp1−/− dependent T cell responses. Peritoneal macrophages were loaded with OVA protein, killed with oxaliplatin for 18 hrs and inoculated into WT versus Casp-1−/− mice. The immune responses were then measured by assessing the capacity of draining lymph nodes cells to produce IFNγ. For a and c, five mice/group were used. One representative experiment out of three showing means±SEM of triplicates is depicted. * p<0.05.

FIG. 12: T cell responses to natural tumor antigens contained in dying tumor cells depend on caspase-1. Identical setting as in FIG. 5d but B16F10 melanoma cells treated with oxaliplatin were used to immunize WT or Casp-1 deficient mice. Five to seven days later, mice (n=5 mice/group) were sacrificed to harvest popliteal lymph nodes for in vitro restimulation with gp100-derived peptide (QVPRNQDWL) and assessment of IFNγ production. * p<0.05.

FIG. 13: The protective antitumor effects of dying tumor cells critically depend on the P2X₇/NAL P3/Casp1 axis. MCA205 cells treated in vitro with mitoxantrone (MTX) were inoculated subcutaneously in the indicated genetic backgrounds of C57BL/6 mice (n=5/group). 7-10 days later, mice were rechallenged with live MCA205 cells. The percentages of tumor free mice are pooled from three independent experiments. *p<0.05.

FIG. 14: The immunogenicity of cell death requires active IL-1β. Mice were immunized with PBS or dying CT26 tumor cells treated with doxorubicin or freeze thawing and LPS. Dying CT26 were co-inoculated together with IL-1Ra (100 μg) into the flank of WT mice. Five days later, mice (n=5 mice/group) were sacrificed to harvest popliteal lymph nodes for in vitro restimulation with CT26 or MCA205 tumor lysate and assessment of IFNγ production. *p<0.05.

FIG. 15. IL-1β-dependent CTL priming by dying tumor cells in vitro and in vivo. FIG. 15a. MyD88 is required at the level of the host but not that of the APC. DC from WT mice were first loaded with antigen (OVA holoprotein with C/P adjuvant, live or oxaliplatin-treated EG7 cells) and then injected into the footpad of into WT or MyD88^{-/-} recipients (n=6 mice/group). Five days later, the local immune response was measured as in FIG. 2d,e. Results of one typical experiment out of three are depicted. FIG. 15b. Casp-1 and IL-1β-dependent OT-I priming in vitro. The differentiation of IFNγ producing CD8⁺ OT-1 cells was induced by incubating naïve OVA-specific TCR transgenic OT-1 lymphocytes with syngeneic BM-DC (derived from WT or Casp-1^{-/-} mice) that were loaded with oxaliplatin-treated EG7 for 2 days in vitro. Antibody-mediated neutralization of IL-1β markedly reduced the levels of IFNγ measured by ELISA in the supernatants of the 2-day coculture (while isotype control antibodies failed to do so). Exogenous addition of rIL-1β (100 ng/ml) or IL-12 (10 ng/ml) to cocultures of Casp-1^{-/-} DC loaded with dying tumor cells and incubated with OT1 restored T cell priming. The inset shows that priming with OVA holoproteins in the presence of TLR agonists is independent of Casp-1. FIG. 15c. Specific and direct polarizing effects of IL-1β on T lymphocytes in vitro. Purified CD3⁺ CD8⁺ T cells derived from naïve WT mice were stimulated with anti-CD3 and anti-CD28 mAb for 5 days in the presence of the indicated cytokines (10 ng/ml for IL-12p70, 100 ng/ml for IL-1β, IL-6 and TNFα) -and IFNγ production was measured as in FIG. 3b. FIG. 15d. The adaptive T cell immune response elicited by oxaliplatin-treated EG7 was abrogated by IL-1R antagonists. Identical setting as in FIG. 5a, but adding IL-1R antagonist (IL-1Ra) to the vaccine. FIG. 15e. Exogenous supply of recombinant IL-1β or IL-12 (but not IL-6) restored T cell priming in mice deficient in the P2X7/NALP3/Casp-1 pathway. Identical setting as in FIG. 2d, but rIL-1β (250 ng/footpad), rIL-12 (50 ng/footpad) or rIL-6 (250 ng/footpad) were co-injected with the dying tumor cells. The in vitro experiments (a, b) were conducted twice and one representative experiment is depicted. Means±SEM of triplicate wells are indicated. Each experimental group contained 4-8 mice in two independent experiments. *p<0.05.

FIG. 16. IL-1R antagonists abrogate the capacity of dying tumor cells to stimulate IFNγ production by CD8+ T cells. Flow cytometry analyses of the CD3⁺CD8⁺ T cells producing IFNγ following restimulation with PMA-ionomycin in vitro, five days after in vivo priming using oxaliplatin-treated tumor cells in the presence or absence of IL-1R antagonists. FIG. 16a. Representative dot plot of IFNγ producing CD8+ T cells. FIG. 16b. Quantification of IFNγ producing CD8+T cells. *p<0.05.

FIG. 17. The anti-cancer activity of oxaliplatin depends on P2X₇ and the NALP3 inflammasome. FIG. 17a,b,c. Therapeutic efficacy of oxaliplatin on established tumors depends on the integrity of the immune system on the P2X₇/NALP3/casp-1 axis. EL4 thymoma tumors were established in syngenic mice bearing the indicated genotypes (a-c) or injected with anti-CD8 antibody (a). When the tumor reached 70-90mm² in size, mice were either left untreated or were treated with oxaliplatin. Each treatment group included 5-6 mice (X±SEM), and each experiment was repeated three times with similar results. * p<0.05. d. Caspase 1 is required for the immune response promoted by oxaliplatin chemotherapy. EG7 tumors established in the thigh were treated with one systemic injection of oxaliplatin. Inguinal lymph node cells were harvested 5 days later, and the gangliocytes were restimulated in vitro. IFNγ secretion was assessed by ELISA. Results (means of triplicates ±SEM, n=3) are representative of one typical experiment out of three. ^{*}P<0.01. FIG. 17d. The efficacy of anthracyclines against established colon cancer is dependent on IL-1β. CT26 tumors established for 10 days in WT mice were treated with one local injection of doxorubicin in the absence or presence of neutralizing anti-IL-1β Ab (n=10/group, *p<0.05). FIG. 17e. A single-nucleotide polymorphism (SNP) in P2X7 (rs3751143) affects the long-term efficacy of conventional anti-cancer therapy in breast cancer patients. Comparative Kaplan-Meier estimates of time to metastasis in two groups of patients bearing the normal (Glu496Glu) or loss-of-function (Glu496Ala) P2X7 alleles. The time to metastatic progression was analyzed in 225 women with non-metastatic breast cancer who received adjuvant chemotherapy with anthracylines.

### DETAILED DESCRIPTION OF THE INVENTION

Immunogenic cell death elicited, for instance, by anthracyclines, oxaliplatin or X-rays is characterized by a pre-apoptotic exposure of calreticulin on the plasma membrane, facilitating the uptake of dying cells by DC and by the post-apoptotic release of HMGB1 which acts on TLR4 present on DC, stimulating the processing of antigens. However, addition of recombinant calreticulin or HMGB1 to live tumor cells is not sufficient to elicit the presentation of their antigens by DC, implying that additional signals must be exchanged between dying cells and DC.

The present invention is based on the observation that the P2X₇-elicited NALP3-inflammasome controls immune responses against dying tumor cells. Indeed, dying tumor cells release ATP, which then acts on P2X₇ purinergic receptors from DC and triggers the NALP3 caspase-1 activation complex, allowing for the secretion of interleukin-1β (IL-1β).

In the absence of the IL-1 receptor 1 or in the presence of IL-1 receptor antagonist, dying tumor cells fail to prime cancer-specific interferon-γ-producing CTL. The inventors have thus identified NALP3 inflammasome-dependent IL-1 β production as a critical element of CTL polarization towards IFNγ production. Until now, chronic inflammation including overactivation of the IL-1β/IL-1βR system (Krelin et al., 2007) has been considered as a tumor-promoting condition, arguing in favour of IL-1β inhibition for tumor prevention or therapy (Hagemann et al., 2007; Greten et al., 2004; Naugler et al., 2007; Balkwill et al., 2005).

Inventors herein also demonstrate that anticancer chemotherapy that was successful in immunocompetent hosts turned out inefficient against tumors established in a subject having a loss of function in the P2X₇-elicited NALP3-inflammasome pathway. They observed that the absence of a functional P2X₇/NALP3/Casp-1/IL-1β axis abolished the IFNγ production and that CTL priming by dying tumor cells fails in such subject unless exogenous IL-1β_{.} is provided.

Furthermore, in clinical studies herein described, inventor's data reveal that after a chemotherapy inducing an immunogenic tumor cell death, the metastasis-free survival of patients with cancer who have a loss of function in the P2X₇-elicited NALP3-inflammasome pathway is shorter than that of patients with normal pathway.

Definitions

The term “cancer” as used herein refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). Typical cancer are X-rays, anthracyclines, cisplatin and/or oxaliplatin sensitive cancer such as breast, stomach, sarcoma, ovarian, endometrium, bladder, cervix uteri, rectum, colon, lung, ORL cancer, paediatric tumours (neuroblastoma, glyoblastoma multiforme), lymphoma, leukaemia, myeloma, seminoma, Hodgkin and malignant hemopathies.

The term “metastatic relapse”, as used herein, refers to the transmission of cancerous cells from the primary tumor to one or more sites elsewhere in a patient who initially responded to previous therapy, but in whom the therapeutic response was not maintained. The metastatic relapse appears after a remission period.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

In particular, the term “to treat a cancer” or “treating a cancer” means reversing, alleviating, inhibiting the progress of, or preventing, either partially or completely, the growth of tumors, tumor metastases, or other cancer-causing or neoplastic cells in a patient.

As used herein, the term “chemotherapeutic treatment” or “chemotherapy” refers to a cancer therapeutic treatment using chemical or biochemical substances, in particular using one or several antineoplastic agents. Preferably, chemotherapy promotes immunogenic tumor cell death. The term “antineoplastic agent” and “chemotherapeutic agent” are used interchangeably and refer to chemical compounds or drugs which are used in the treatment of cancer. Preferably, the chemotherapy involves the use of at least one antineoplastic agent selected from the group consisting of anthracyclines such as Aclarubicin, Daunorubicin, Doxorubicin, Epirubicin, Idarubicin, Amrubicin, Pirarubicin, Valrubicin, Zorubicin, Carminomycin and Detorubicin; platinum-based chemotherapy drugs such as Carboplatin, Cisplatin, Nedaplatin, Oxaliplatin, Triplatin tetranitrate and Satraplatin; anthracenediones such as Mitoxantrone and Pixantrone; and antitumor agents isolated from Streptomyces species such as Actinomycin, Bleomycin, Mitomycin and Plicamycin, and derivatives thereof. More preferably, the chemotherapy is selected from the group consisting of anthracyclines, oxaliplatin and cisplatin.

The term “radiotherapeutic treatment” or “radiotherapy” is a term commonly used in the art to refer to multiple types of radiation therapy including internal and external radiation therapy, radioimmunotherapy, and the use of various types of radiation including X-rays, gamma rays, alpha particles, beta particles, photons, electrons, neutrons, radioisotopes, and other forms of ionizing radiation. Preferably, the radiotherapy involves the use of X-rays or gamma-rays.

Chemotherapy and radiotherapy can be used alone or in combination.

The term “anticancer treatment inducing immunogenic cell death” refers to chemotherapeutic or radiotherapeutic treatment which induces a modality of cell death that is apoptotic in morphology and highly efficient in eliciting an immune response, in the absence of any adjuvant. Anticancer treatments inducing immunogenic cell death may be, but not limited to, anthracyclines, oxaliplatin, cisplatin and X-rays.

The term “sensitivity to a treatment” refers to the level of response of a subject to a treatment, including but not limited to the ability to metabolize a therapeutic compound, to the ability to convert a pro-drug to an active drug, to the pharmacokinetics (absorption, distribution, elimination) and to the pharmacodynamics (receptor-related) of a drug in an individual.

The term “resistance to a treatment” refers to an innate or acquired condition in which the subject does not respond to the treatment. If the resistance is acquired, the subject initially responds to the treatment but the cancer relapses within six months of completing the initial treatment.

As used herein, the term “subject” preferably refers to a human in need of treatment to treat cancer, including adults, children and at the prenatal stage. However, the term “subject” can also refer to non-human animals, preferably mammals such as dogs, cats, horses, cows, pigs, sheep and non-human primates, among others, that are in need of treatment.

As used herein, the term “P2X₇-elicited NALP3 inflammasome pathway” refers to the metabolic axis comprising the P2X₇ receptor (UniGene Hs.507102) the NALP3 protein (UniGene Hs.159483), the adaptator protein ASC (UniGene Hs.499094), the caspase-1 (UniGene Hs.2490) and IL-1β (UniGene Hs.126256).

P2X₇ receptors are a family of cation-permeable ligand gated ion channels that open in response to the binding of extracellular adenosine 5′-triphosphate (ATP). They belong to a larger family of receptors known as the purinergic receptors.

The NALP3 inflammasome is a multiprotein complex that is responsible for the activation of caspase-1 leading to the processing and secretion of cytokines IL-1β and IL-18. The NALP3 inflammasome is composed of NALP3 protein which belongs to the NLR family of cytoplasmic proteins, caspase-1 and the adaptator protein ASC which connects NALP proteins to the caspase-1.

As used herein, the term “functional status” of a pathway refers to the ability or the disability of a pathway to fulfill its function. The functional status of the P2X₇-elicited NALP3-inflammasome pathway depends on the expression and the activity of all proteins comprised in this pathway, i.e. P2X₇, NALP3, ASC, Caspase-1 and IL-1β. The P2X₇-elicited NALP3-inflammasome pathway is non-functional if dendritic cells of the subject do not secrete IL-1β or in a reduced amount by comparison with a standard level.

The term “loss of function” of a component of the pathway, as used herein, refers to a reduced activity or the absence of activity of this component.

The term “functional analysis” of a protein, as used herein, refers to the analysis of the function of a protein by assessing its activity.

As used herein, the term “mutation” encompasses point mutation, deletion, rearrangement and/or insertion in the coding and/or non-coding region of the locus, alone or in various combination(s). Deletions may encompass any region of one, two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The mutation may result in the creation of stop codons, frameshift mutations, amino acid substitutions, particular RNA splicing or processing, product instability, truncated polypeptide production, etc. The mutation may result in the production of a polypeptide with altered function, stability, targeting or structure. It may also cause a reduction in protein expression or, alternatively, an increase in said production.

The term “loss-of-function mutation” refers to a mutation which affects the activity of the protein encoded by the mutated gene. The protein may be less active or completely inactive.

The term “loss of function in the P2X₇-elicited NALP3-inflammasome pathway” means that a gene encoding a protein comprised in the P2X₇-elicited NALP3-inflammasome pathway as defined above carries a loss-of-function mutation or that a protein of the pathway is less active or completely inactive or that a protein of the pathway is less expressed or not expressed. This loss of function results in a reduced capacity or incapacity of dendritic cells to secrete IL-1β.

The term “SNP” means“single nucleotide polymorphism”. SNP can be referred by the location of the amino acid residue which is modified due to the polymorphism (e.g. Arg307Gln) or by the reference SNP number (e.g. rs28360457) of the NCBI SNP database (http ://www.ncbi.nlm.nih. gov/SNP).

By a “therapeutically effective amount” is intended an amount administered to a subject that is sufficient to elicit the biological or medical response of a tissue, system, animal or human that is being sought by the researcher, veterinarian, medical doctor or other clinician.

The term “standard level” or “standard value”, as used herein, refers to a level or a value which is obtained from a population of individuals having a functional P2X₇-elicited NALP3-inflammasome pathway or from cells provided by these individuals.

Efficient anticancer responses depend on the capacity of dendritic cells to present antigen from dying cancer cells and to prime tumor-specific cytotoxic T lymphocytes (CTL). As described in the experimental section, the inventors have demonstrated that NALP3 inflammasome-dependent IL-1β production is a critical element of CTL polarization towards IFNγ production and thus, of efficient anticancer response.

In a first aspect, the present invention provides an in vitro method of assessing the sensitivity of a subject to a chemotherapeutic or radiotherapeutic treatment of cancer, wherein the method comprises determining the functional status of the P2X₇-elicited NALP3 inflammasome pathway, a non-functional P2X₇-elicited NALP3 inflammasome pathway being indicative of a resistance to said treatment.

In a first embodiment, the functional status of the P2X₇-elicited NALP3 inflammasome pathway is assessed by the detection of a loss-of-function mutation in a gene involved in said pathway, the presence of said mutation being indicative of a non-functional P2X₇-elicited NALP3 inflammasome pathway. This mutation may be comprised in a gene encoding P2X₇, NALP3, ASC, Caspase-1 or IL-1β, or in an expression regulating element such as a promoter.

A loss-of-function mutation can be detected by any means known by the man skilled in the art, for instance by sequencing all or part of P2X₇, NALP3, ASC, Caspase-1 or IL-1β gene, using selective hybridization and/or amplification of all or part of these genes or restriction digestion. More preferably, a specific amplification of the gene is carried out before the mutation identification step. The sequencing may be performed on specific domains, typically those known or suspected to carry deleterious mutations.

The loss-of-function mutation can also be detected at the RNA level, for instance, by detecting a RNA mutated sequence or an abnormal RNA splicing or processing or level of expression. This may be carried out by various techniques known in the art, including restriction digestion, sequencing of all or part of the RNA of interest, selective hybridization or selective amplification of all or part of said RNA.

Preferably, the loss-of-function mutation to be detected is a single nucleotide polymorphism (SNP) comprised in a gene encoding P2X₇, NALP3, ASC, Caspase-1 or IL-1β, or in an expression regulating element of these genes.

The functional status of the P2X₇-elicited NALP3 inflammasome pathway may be assessed by detecting a loss-of-function SNP in the gene encoding P2X₇. Preferably, the SNP is selected from the group consisting of Arg307Gln (rs28360457; SEQ ID No. 2), Ileu568Asn (rs1653624; SEQ ID No. 3), Glu496Ala (rs3751143; SEQ ID No. 1) and Thr357Ser (rs2230911; SEQ ID No. 4). More preferably, the SNP is Glu496Ala (rs3751143).

The functional status of the P2X₇-elicited NALP3 inflammasome pathway may also be assessed by detecting a loss-of-function SNP in the gene encoding caspase-1. Preferably, the SNP is rs501192 (SEQ ID No. 5).

Amplification may be performed according to various techniques known in the art, such as polymerase chain reaction (PCR), ligase chain reaction (LCR), strand displacement amplification (SDA), nucleic acid sequence based amplification (NASBA) and restriction fragments length polymorphism (RFLP). Preferably, allele-specific PCR or PCR-SSCP is used. Suitable primers to perform these amplification reactions are easily designed by the skilled person based on sequence information found in databases, such as Genbank.

Restriction digestions, sequencing reactions, selective hybridization and selective amplification may be carried out according to well-known protocols such as those described in Sambrook et al. (Sambrook et al. Molecular Cloning, A Laboratory Manual, Third Edition, Cold Spring Harbor Laboratory Press, 2000) and in Ausubel et al. (In Current Protocols in Molecular Biology, John Wiley & Sons, NY, 1998).

In another embodiment, the functional status of the P2X₇-elicited NALP3 inflammasome pathway is assessed by the detection of a mutated polypeptide sequence or an impaired expression of a protein involved in said pathway, a mutated sequence or an impaired expression being indicative of a non functional P2X₇-elicited NALP3 inflammasome pathway. The protein involved in the P2X₇-elicited NALP3 inflammasome pathway may be selected from the group consisting of P2X₇, NALP3, ASC, Caspase-1 and IL-1β.

A mutated polypeptide sequence can be detected by various techniques known in the art such as, for example, polypeptide sequencing and/or binding to specific ligands such as antibodies.

An impaired expression of a protein involved in the P2X₇-elicited NALP3 inflammasome pathway can also be detected by various techniques known in the art such as, for instance, Western-blot, ELISA, radio-immunoassay or immuno-enzymatic assays. The expression of the protein can also be assessed by detecting its specific RNA expression by using northern-blot or quantitative RT-PCR, or any other method known in the art.

Other suitable methods may be used to detect of a mutated polypeptide or polynucleotide sequence or to quantify the protein expression or a RNA. They include, without limitation, allele-specific oligonucleotide (ASO), allele-specific amplification, Southern-blot, single-stranded conformation analysis, PFGE, fluorescent in situ hybridization (FISH), gel migration, clamped denaturing gel electrophoresis, heteroduplex analysis, RNase protection, and chemical mismatch cleavage.

In another embodiment, the functional status of the P2X₇-elicited NALP3 inflammasome pathway is assessed by comparing the IL-1β level in a blood sample of the subject before and after a chemotherapeutic or radiotherapeutic treatment, a significant increase of said level after said treatment being indicative of a functional P2X₇-elicited NALP3 inflammasome pathway. Preferably, the treatment is selected from the group consisting of anthracyclines, oxaliplatin, cisplatin and X-rays.

As used herein, the term “significant increase” refers to an increase that is statistically comparable to those obtained in the same condition in subjects having a functional P2X₇-elicited NALP3 inflammasome pathway.

The treatment administered to the subject generates dying tumor cells which induce, in a subject having a functional P2X₇-elicited NALP3 inflammasome pathway, an increased IL-1β secretion by dendritic cells and thus an increased IL-1β level in the blood. If the subject does not have a functional P2X₇-elicited NALP3 inflammasome pathway, no increase in IL-1β blood level is observed or a lower level of it. IL-1β may be measured by any method known by the skilled person, for instance using an ELISA assay.

In a preferred embodiment, IL-1β level is measured in a blood serum sample of the subject before and 24 and/or 72 hours after the chemotherapeutic or radiotherapeutic treatment.

Consequently, with this method, the sensitivity of a subject to an anticancer treatment may be assessed after the first administration by only measuring IL-1β concentration in a blood sample of this subject.

In a further embodiment, the functional status of the P2X₇-elicited NALP3 inflammasome pathway is assessed by measuring the capacity of the dendritic cells (DC) of the subject to secrete IL-1β in presence of dying tumor cells, wherein a reduced capacity compared to a standard level is correlated to a non-functional P2X₇-elicited NALP3 inflammasome pathway.

This capacity of secretion may be assessed, for example, with the method comprising the step of (i) obtaining DC from a sample of the subject, (ii) co-cultivating said cells with tumor cells treated with an antineoplastic agent, (iii) assessing IL-1β in the cell culture supernatant, wherein a decreased level of IL-1β compared to a standard level is correlated to a non-functional P2X₇-elicited NALP3 inflammasome pathway.

The standard level is obtained by submitting dendritic cells from subjects having a functional P2X₇-elicited NALP3 inflammasome pathway to the same protocol.

As negative control, DC may be incubated alone, or with living tumor cells.

Preferably, autologous DC are obtained from a blood sample of the subject. Firstly, CD14+ monocytes are harvested from peripheral blood mononuclear cells (PBMC) after a positive cell sorting using anti-CD14 antibodies or an adherence step on plastic dishes, or by any other method known in the art. Then, CD14+ monocytes are incubated four days with compounds which stimulate dendritic cell differentiation such as GM-CSF and IFNα2b.

Tumor cells used in this method have to be sensitive to the antineoplastic agent and may be easily chosen by the skilled person. For example, HCT116 cells (colon carcinoma) treated with oxaliplatin may be used. These cells are added to DC culture without any washing step.

Preferably the antineoplastic agent is selected from the group consisting of anthracyclines, oxaliplatin, cisplatin and X-rays.

An example of an embodiment of this method is presented in the experimental section.

In another embodiment, the functional status of the P2X₇-elicited NALP3 inflammasome pathway is assessed by measuring the capacity of the dendritic cells (DC) of the subject to secrete IL-1β in presence of HMGB1 and ATP, wherein a reduced capacity compared to a standard level is correlated to a non-functional P2X₇-elicited NALP3 inflammasome pathway.

As used herein, the term “HMGB1” refers to the high-mobility group box 1 protein which is a TLR4 agonist released by dying tumor cells treated with antineoplastic agents such as doxorubicin or oxaliplatin (Apetoh et al., 2007).

This method comprises the step of (i) obtaining DC from a sample of the subject, (ii) cultivating said cells with HMGB 1 and ATP, (iii) assessing IL-1β in the cell culture supernatant, wherein a decreased level of IL-1β compared to a standard level is correlated to a non-functional P2X₇-elicited NALP3 inflammasome pathway.

The standard level is obtained by submitting dendritic cells from subjects having a functional P2X₇-elicited NALP3 inflammasome pathway to the same protocol.

As negative control, DC may be incubated without HMGB1 nor ATP.

Preferably, autologous DC are obtained from PBMC-derived monocytes, as described above.

In a further embodiment, the functional status of the P2X₇-elicited NALP3 inflammasome pathway is assessed by measuring the capacity of the monocytes of the subject to secrete IL-1β in presence of lipopolysaccharide and ATP, wherein a reduced capacity compared to a standard level is correlated to a non-functional P2X₇-elicited NALP3 inflammasome pathway

This capacity may be assessed, for example, with the method comprising the step of (i) obtaining CD14+ monocytes from a sample of the subject, (ii) cultivating said cells with lipopolyssacharide and ATP, (iii) assessing IL-1β in the cell culture supernatant, wherein a decreased level of IL-1β compared to a standard level is correlated to a non-functional P2X₇-elicited NALP3 inflammasome pathway.

Preferably, autologous CD14+ monocytes are harvested from peripheral blood mononuclear cells of the subject as described above.

The standard level is obtained by submitting monocytes from subjects having a functional P2X₇-elicited NALP3 inflammasome pathway to the same protocol.

As negative control, monocytes may be incubated without LPS nor ATP.

IL-1β may be assessed in cell culture supernatants by any method known in the art, preferably using ELISA assay. IL-1β may also be assessed with Western-blot analysis using specific antibodies recognizing precursor and mature forms of IL-1β and/or caspase-1, as described below.

In a further embodiment, the functional status of the P2X₇-elicited NALP3 inflammasome pathway is assessed by the functional analysis of components of said pathway, a loss of function of at least one component of said pathway being indicative of a non functional P2X₇-elicited NALP3 inflammasome pathway.

The activity of P2X₇, NALP3, ASC, Caspase-1 and/or IL-1β is assessed to detect any loss of function affecting one of these proteins. The activity of P2X₇, NALP3, ASC, Caspase-1 or IL-1β may be assessed by any method known by the skilled person.

Preferably, the functional status of the P2X₇-elicited NALP3 inflammasome pathway is assessed by the functional analysis of the P2X₇ receptor. As example, the functional analysis of the P2X₇ receptor may be carried out by measuring ATP-induced ⁸⁶Rb⁺ efflux in erythrocytes as described in Sluyter et al. (Sluyter et al., 2004). This analysis of the P2X₇ receptor may be performed on a blood sample of the subject. Obtained values are compared with those obtained with a control subject, i.e. having a functional P2X₇-elicited NALP3 inflammasome pathway. An impaired ATP-induced ⁸⁶Rb⁺ efflux means that the subject has a non-functional P2X₇-elicited NALP3 inflammasome pathway and thus exhibits a reduced sensitivity to an anticancer treatment. As a further example, the functional analysis of the P2X₇ receptor may be carried out by testing monocytes, lymphocytes or macrophages for Ca²⁺, Ba²⁻ or ethidium uptake or influx as described in the article of Jursik et al. (Jursik et al., 2007).

The activity of IL-1β and Caspase-1 may be assessed by investigating their maturation process.

IL-1β is produced as a biologically inactive precursor, i.e. pro-IL-1β, which is converted to the active form, i.e. IL-1β p17, by proteolytic processing achieved by Caspase-1, also termed IL-1β-converting enzyme (ICE).

Caspase-1 is synthesized as a proenzyme of 45 kDa, which undergoes proteolytic cleavage to produce maturation products, i.e. p20 and p10 subunits, which heterodimerize to form the active protease.

Precursor and mature forms of caspase-1 and IL-1β may be detected by Western-blot using specific antibodies recognizing these proteins in the supernatants and the lysates of PBMC-derived monocytes stimulated with ATP+LPS or DC (generated from PBMC-derived monocytes as described above) incubated with dying tumor cells (e.g. HCT116 cells incubated with oxaliplatin and not washed prior to incubation with DC) or DC incubated with HMGB1 and ATP.

If the P2X₇-elicited NALP3 inflammasome pathway is functional, caspase-1 p20 is detected in cellular lysates and IL-1β p17 is detected in cell culture supernatants.

If the P2X₇-elicited NALP3 inflammasome pathway is not functional, IL-1β p17 is not detected in cell culture supernatants. This could be resulted from the absence of caspase-1 and/or IL-1β precursor expression or from a mutation inducing an abnormal maturation of these precursors.

The specificity of the IL-1β produced following inflammasome activation may be assayed by running the above described experiments with or without antagonists of caspase-1, such as high concentrations of KCl (e.g. 130 mM) or z-VAD-fmk. In presence of caspase-1 antagonist, IL-1β production by stimulated monocytes or DC is abolished even if the P2X₇-elicited NALP3 inflammasome pathway is functional.

In a second aspect, the present invention provides an in vitro method for screening a compound useful for increasing or restoring the sensitivity to a chemotherapeutic or radiotherapeutic treatment of cancer in a subject having a non-functional P2X₇-elicited NALP3 inflammasome pathway, wherein the method comprises determining the ability of a test compound to induce or increase IL-1β secretion by dendritic cells in presence of dying tumor cells in said subject.

The present invention further provides an in vitro method for screening a compound useful for treating a cancer in a subject having a non-functional P2X₇-elicited NALP3 inflammasome pathway, wherein the method comprises determining the ability of a test compound to induce or increase IL-1β secretion by dendritic cells in presence of dying tumor cells in said subject.

A compound which is able to induce or increase IL-1β secretion by dendritic cells in presence of dying tumor cells allows restoring or increasing the priming of tumor-specific cytotoxic T lymphocytes. This compound may thus be used to increase or restore the sensitivity to an anticancer treatment in a subject and thus, to treat a cancer in said subject. Preferably, the anticancer treatment is selected from the group consisting in anthracyclines, oxaliplatin, cisplatin and X-rays.

In an embodiment, the ability of a test compound to induce or increase IL-1β production in a subject having a non-functional P2X₇-elicited NALP3 inflammasome pathway is assessed by obtaining DC from a sample of the subject and contacting said cells with dying tumor cells in presence of the test compound and measuring the amount of IL-1β secreted in the cell culture supernatant, wherein a reduced amount of IL-1β compared to a standard level is correlated to a non-functional P2X₇-elicited NALP3 inflammasome pathway.

The standard level of IL-1β secretion is obtained by contacting DC of a subject having a functional P2X₇-elicited NALP3 inflammasome pathway with dying tumor cells in presence of the test compound and measuring the amount of IL-1β secreted in the cell culture supernatant.

As negative control, contacting said DC with dying tumor cells in absence of the test compound may be used.

As described above, a non-functional P2X₇-elicited NALP3 inflammasome pathway 5 may result from the loss of function of different components of said pathway. Thus, DC provided from different subjects having a non-functional pathway may exhibit different responses to a test compound according to the component of the pathway afflicted by the loss of function. Consequently, dendritic cells used in this screening method are preferably provided from the subject to be treated.

The present invention further provides a method for screening a compound useful for increasing or restoring the sensitivity to a chemotherapeutic or radiotherapeutic treatment of cancer in a subject having a non-functional P2X₇-elicited NALP3 inflammasome pathway, wherein the method comprises (i) administering a test compound in combination with a chemotherapeutic or radiotherapeutic treatment of cancer to a non-human transgenic animal with non-functional P2X₇-elicited NALP3 inflammasome pathway and inoculated with a tumor, and (ii) assessing the sensitivity of said animal to said treatment.

Preferably, the non-human transgenic animal with non-functional P2X₇-elicited NALP3 inflammasome pathway is selected from the group consisting in BALB/c and C57BL/6 mice exhibiting a Casp1^{-/-}, Asc^{-/-}, Nalp3^{-/-}, IL-1β^{-/- or P}2X₇^{-/-} genotype. As example, C57BL/6 Casp1^{-/-}, Asc^{-/-}, Nalp3^{-/-}, P2X₇^{-/-} mice were described in Li et al., 1995; Srinivasula et al., 2002; Kanneganti et al., 2006; Solle et al., 2001; respectively.

Tumor cells used to inoculate a tumor to the non-human transgenic animal are preferably selected from the group consisting of CT26 colon cancer cells, EL4 thymoma cells, EG7 cells, MCA205 fibrosarcoma cells, B16F10 melanoma cells and TS/A mammary adenocarcinoma cells.

The sensitivity of the non-human transgenic animal to the administered treatment may be assessed by any method known in the art such as tumor size follow-up or metastasis free survival of animals.

Inventors' data presented in the experimental section, demonstrate that the probability of metastasis relapse is increased in a subject having a loss of function in the P2X₇-elicited NALP3 inflammasome pathway.

The present invention thus provides an in vitro method for determining the likelihood of a metastatic relapse in a subject, which method comprises determining the functional status of the P2X₇-elicited NALP3 inflammasome pathway in said subject, a non-functional pathway being indicative of an increase likelihood of a metastatic relapse.

The functional status of the P2X₇-elicited NALP3 inflammasome pathway may be assessed by any method as described above.

In a further aspect, the invention provides an in vitro method for selecting the proper chemotherapeutic or radiotherapeutic treatment for a subject in need thereof, wherein the method comprises determining the functional status of the P2X₇-elicited NALP3 inflammasome pathway, a non-functional pathway being considered as a contraindication for an anticancer treatment inducing immunogenic tumor cell death.

The term “contraindication” as used herein, refers to an imbalance between the side effects of the therapeutic treatment and the response of the patient to said treatment. In this case, the treatment induces more detrimental effects than beneficial effects.

If the subject has a functional P2X₇-elicited NALP3 inflammasome pathway, he may be sensitive to an anticancer treatment inducing immunogenic tumor cell death such as anthracyclines, oxaliplatin, cisplatin or X-rays.

On the contrary, if the subject has a non-functional P2X₇-elicited NALP3 inflammasome pathway, an anticancer treatment inducing immunogenic tumor cell death will have a reduced therapeutic effect.

Consequently, determining the functional status of the P2X₇-elicited NALP3 inflammasome pathway allows the practitioner to determine if an anticancer treatment inducing immunogenic tumor cell death is suitable to treat a subject or if other treatments have to be considered; or to provide information allowing the practitioner to decide what is the appropriate treatment.

The functional status of the P2X₇-elicited NALP3 inflammasome pathway may be assessed by any method as described above.

The present invention also concerns a pharmaceutical composition comprising a chemotherapeutic agent and a compound which is able to compensate a non-functional P2X₇-elicited NALP3 inflammasome pathway.

In a particular embodiment, the chemotherapeutic agent is selected from the group consisting of anthracyclines, oxaliplatin and cisplatin.

The compound which is able to compensate a non-functional P2X₇-elicited NALP3 inflammasome pathway may be screened by a screening method of the invention.

In an embodiment, this compound is selected from the group consisting of IL-1β; IL-12; natural killer cells and natural killer T cells activators such as CD1d agonists, IL-15, IL-2 or IFNα; P2X₇ receptor activators such as the cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies and IL-33. Antibodies may be neutralizing or blocking and molecules are preferably provided in their active form such as IL-1β p17 for IL-1β and IL-12 p70 for IL-12.

In a preferred embodiment, the compound is recombinant IL-1β.

The pharmaceutical composition comprising a chemotherapeutic agent and a compound which is able to compensate a non-functional P2X7-elicited NALP3 inflammasome pathway is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, rectal, topical (including transdermal, buccal and sublingual), parenteral (including subcutaneous, intramuscular, intravenous, intra-arterial and intradermal) or intra-tumoral (e.g. ultrasound-guided intratumoral injection) administration. Intra-tumoral administration may be contemplated for localized non metastatic tumor. For these formulations, conventional excipient can be used according to techniques well known by those skilled in the art.

The compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In a preferred embodiment, the pharmaceutical composition of the invention is suitable for parenteral or oral administration.

Pharmaceutical composition according to the invention may be formulated to release the active drugs substantially immediately upon administration or at any predetermined time or time period after administration.

Pharmaceutical composition according to the invention can comprise one or more chemotherapeutic agents and one or more compounds which are able to compensate a non-functional P2X7-elicited NALP3 inflammasome pathway, associated with pharmaceutically acceptable excipients and/or carriers. These excipients and/or carriers are chosen according to the form of administration as described above.

The present invention also concerns a product containing a chemotherapeutic agent and a compound which is able to compensate a non-functional P2X₇-elicited NALP3 inflammasome pathway, as a combined preparation for simultaneous, separate or sequential use in the treatment of cancer, in particular in a subject having a non functional P2X₇-elicited NALP3 inflammasome pathway.

The amount of chemotherapeutic agent and of compound which is able to compensate a non-functional P2X₇-elicited NALP3 inflammasome pathway has to be determined by standard procedure well known by those of ordinary skill in the art. Physiological data of the patient (e.g. age, size, and weight) and the routes of administration have to be taken into account to determine the appropriate dosage.

In a preferred embodiment, the pharmaceutical composition comprises a therapeutically effective amount of chemotherapeutic agent and a therapeutically effective amount of a compound which is able to compensate a non-functional P2X₇-elicited NALP3 inflammasome pathway.

Suitable means and measures to determine the therapeutically effective amount are available to the person skilled in the art.

The present invention further provides a compound which is able to compensate a non-functional P2X₇-elicited NALP3 inflammasome pathway, for use in the treatment of a cancer, in combination with a chemotherapeutic agent or a radiotherapeutic treatment, in particular in a subject having a non-functional P2X₇-elicited NALP3 inflammasome pathway.

This compound may be screened according to a screening method of the invention as described above.

In a particular embodiment, the compound which is able to compensate a non-functional P2X₇-elicited NALP3 inflammasome pathway is selected from the group consisting of IL-1β; IL-12; natural killer cells and natural killer T cells activators such as CD1d agonists, IL-15, IL-2 or IFNα; P2X₇ receptor activators such as the cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies, and IL-33. Antibodies may be neutralizing or blocking and molecules are preferably provided in their active form such as IL-1β p17 for IL-1β and IL-12 p70 for IL-12.

In a preferred embodiment, this compound is recombinant IL-1β.

In another aspect, the invention concerns a compound which is able to compensate a loss of function in the P2X₇-elicited NALP3 inflammasome pathway, for the manufacture of a medicament for treating cancer in a subject having a loss of function in the P2X₇-elicited NALP3 inflammasome pathway. This compound may be screened by a method of the invention as described above

In a particular embodiment, the compound which is able to compensate a loss of function in the P2X₇-elicited NALP3 inflammasome pathway is selected from the group consisting of IL-1β; IL-12; natural killer cells and natural killer T cells activators such as CD1d agonists, IL-15, IL-2 or IFNα; P2X₇ receptor activators such as the cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies, and IL-33. Antibodies may be neutralizing or blocking and molecules are preferably provided in their active form such as IL-1β p17 for IL-1μ and IL-12 p70 for IL-12.

In a preferred embodiment, this compound is recombinant IL-1β.

The present invention also concerns a method for increasing the efficacy of a chemotherapeutic or radiotherapeutic treatment in a subject suffering from a cancer and having a non-functional P2X₇-elicited NALP3 inflammasome pathway, wherein the method comprises administering a chemotherapeutic or radiotherapeutic treatment in combination with a therapeutically effective amount of compound which is able to compensate a non-functional P2X₇-elicited NALP3 inflammasome pathway.

The compound which is able to compensate a non-functional P2X₇-elicited NALP3 inflammasome pathway, and the chemotherapeutic or radiotherapeutic treatment may be administered simultaneously, separately or sequentially.

In a particular embodiment, the compound which is able to compensate a non-functional P2X₇-elicited NALP3 inflammasome pathway is selected from the group consisting of IL-1μ; IL-12; natural killer cells and natural killer T cells activators such as CD1d agonists, IL-15, IL-2 or IFNα; P2X₇ receptor activators such as the cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies, and IL-33. Antibodies may be neutralizing or blocking and molecules are preferably provided in their active form such as IL-1β p17 for IL-1β and IL-12 p70 for IL-12.

Preferably this compound is recombinant IL-1β.

In a preferred embodiment, the method comprises administering an anticancer treatment selected from the group consisting of anthracyclines, oxaliplatin, cis-platin and X-rays in combination with recombinant IL-1β.

The present invention further provides a method for treating a subject suffering from a cancer and having a non-functional P2X₇-elicited NALP3 inflammasome pathway, wherein the method comprises administering a chemotherapeutic or radiotherapeutic treatment in combination with a compound which is able to compensate a non-functional P2X₇-elicited NALP3 inflammasome pathway.

The compound and the anticancer treatment, chemotherapy or radiotherapy, may be administered simultaneously, separately or sequentially. Preferably, the compound which is able to compensate a non-functional P2X₇-elicited NALP3 inflammasome pathway is administered the same day as the chemotherapeutic or radiotherapeutic treatment and during several days after this treatment, e.g. during about two, three, four days or one week. This protocol is repeated at each cycle of treatment.

In a particular embodiment, the compound which is able to compensate a non-functional P2X₇-elicited NALP3 inflammasome pathway is selected from the group consisting of IL-1β; IL-12; natural killer cells and natural killer T cells activators such as CD1d agonists, IL-15, IL-2 or IFNα; P2X₇ receptor activators such as the cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies, and IL-33. Antibodies may be neutralizing or blocking and molecules are preferably provided in their active form such as IL-1β p17 for IL-1β and IL-12 p70 for IL-12. Preferably this compound is recombinant IL-1β.

In a preferred embodiment, the method comprises administering an anticancer treatment selected from the group consisting of anthracyclines, oxaliplatin, cis-platin and X-rays in combination with recombinant IL-1β.

The following examples are given for purposes of illustration and not by way of limitation.

### EXAMPLES

Material and Methods

Mouse Strains

BALB/c (H-2^{d}), C57BL/6 (H-2^{b}), nu/nu and Rag2^{-/-} C57BL/6 mice were obtained from the Centre d'élevage Janvier (Le Genest St Isle, France) and from Charles River Laboratories (L'Arbresle, France), as well as from Taconic (Denmark).

C57BL/6 Ifnγr1^{-/-}, Casp1^{-/-}, Asc^{-/-}, Nalp3^{-/-}, P2X₇^{-/-}and IL-12Rβ2^{-/-} mice were described in Huang et al, 1993; Li et al., 1995; Srinivasula et al., 2002; Kanneganti et al., 2006; Solle et al., 2001; and Wu et al., 2000, respectively. Mice were bred in pathogen-free conditions. Animals were used between 6 and 20 weeks of age. All animals were maintained according to both the FELASA guidelines and the Animal Experimental Ethics Committee Guidelines (Val de Marne, France).

Reagents and Materials

Cell death was induced either with doxorubicin, mitoxantrone (Sigma Aldrich, St Quentin Fallavier, France), or oxaliplatin (Sanofi-Aventis, France). Ovalbumin protein was purchased from Calbiochem (Darmstadt, Germany), ionomycin phorbol 12-myristate 13-acetate (PMA), antimycin A, 2 deoxyglucose, ATP and oxidized ATP (OxiATP) from Sigma Aldrich. Recombinant human HMGB1, mouse IL-1β, IL-6, IL-12 and TNFα and anti-IL-1β neutralizing antibody were obtained from R&D Systems (Lille, France). In some experiments, HMGB1 activity was neutralized with an anti-HMGB1 antibody provided by Huan Yang (Lexington, Mass.) (Huston et al., 2008). IL-1Ra, Kineret Anakinra (Amgen), was kindly provided by the CNRS (Orleans, France) and by the Cochin University Hospital (Paris, France) (Fleischmann et al., 2004). Monoclonal fluorochrome conjugated anti-mouse CD3, CD4, CD8, IFNγ, CD11c, I-A^{b}, CD40, CD80, CD86, control isotypes antibodies, Cytofix/Cytoperm, brefeldin A, and Quantikine ELISA kits for IFNγ, IL-12p40 and IL-10 were purchased from BD Pharmingen (Le Pont de Claix, France). The IL-1β ELISA kit was purchased from Clinisciences (Montrouge, France). IL-17 and IL-4 ELISA kits were purchased from Ozyme (Paris, France). The IL-13 ELISA kit was purchased from Peprotech (Neuilly sur-Seine, France). Rabbit anti-caspase 1 p20 polyclonal antibody for immunofluorescence microscopy was obtained from Santa Cruz Biotechnology (Santa Cruz, USA). Mouse Anti CD11c Alexa Fluor 488 was purchased from Biolegend (San Diego, USA). SIINFEKL and gp100 (KVPRNQDWL) peptides were obtained from Eurogentec (Seraing, Belgium). CpG ODN 28 was kindly provided by the Pitié Salpétrière University Hospital (Paris, France) (Carpentier et al., 2006). Poly I:C was purchased from Amersham (Buckinghamshire, UK) and LPS was bought from Invivogen (San Diego, Calif.). For CD8⁺ T cell depletion, C57BL/6 mice were intraperitoneally (i.p.) injected with 0.2 ml PBS containing 0.3 mg/mouse of purified IgG rat anti-mouse anti-CD8 mAb (prepared from the 2.43 hybridoma). The depletion of CD4⁺ cells was achieved by injecting mice i.p. with rat anti-mouse anti-CD4 mAb prepared from the GK1.5 hybridoma (0.2 mg/mouse). To eliminate NK cell activity, BALB/c mice were injected i.p. with 30 μl of anti-asialo-GM1 antibody purchased from Wako (Neuss, Germany; antibody titer 1:1000) at day −3, day 0 and day 3 after the start of chemotherapy.

Tumor Cell Lines and Transplantable Tumors

CT26 colon cancer cells (from BALB/c), EL4 thymoma cells (from C57BL/6), EG7 cells (an OVA-transfected EL4 cells), MCA205 fibrosarcoma cells (from C57BL/6), B16F10 melanoma cells (from C57BL/6), TS/A mammary adenocarcinoma cells (from BALB/c) and TS/A-OVA cells (OVA-transfected TS/A cells) were cultured at 37° C. under 5% CO₂ in endotoxin-free RPMI 1640 medium supplemented with 10% FCS, penicillin, streptomycin, 1 mM pyruvate and 10 mM Hepes. Mouse embryonic fibroblasts (MEF) and OVA transfected MEF were cultured in endotoxin-free DMEM medium supplemented with 10% FCS, penicillin, streptomycin, 1 mM pyruvate and 10 mM Hepes. B3Z clones were described in Karttunen et al., 1992.

Bone Marrow—Derived DC and T Cell Hybridoma Assays

Bone marrow—derived dendritic cells (DC) were propagated in Iscoves's medium (Sigma Aldrich) supplemented with Penicillin (100 U/ml Gibco), Streptomycin (100 μg/ml Gibco), L-glutamine (Gibco), 2-mercaptoethanol (50 μM, Sigma), 10% heat-inactivated and filtered, endotoxin-free FCS (Gibco), and 30% of J558 supernatant as previously described (Lutz et al., 1999). DC were used between day 10 and day 12 when the proportion of DC within the culture was above 80%, as determined by simultaneous immunofluorscence detection of CD11c and MHC class II antigen. The SIINFEKL-specific, H-2K^{b}-restricted hybridoma B3Z (2.10⁴ cells per well containing 200 μl culture medium) was cultured in the presence of live or oxaliplatin-treated EG7 cells (which were used 24 hrs after oxaliplatin treatment, 1×10⁴ cells per well) plus WT or loss-of-function DC (1.10⁴ cells per well) (Apetoh et al., 2007). Supernatants were harvested 48 hours later, and IL-2 secretion was assessed by ELISA. As a positive control, the SIINFEKL peptide (2 μg/ml) was used.

Priming Assays

1.10⁶ CT26, EG7, or B16F10 cells were either left untreated or treated with doxorubicin (20 μM) or oxaliplatin (5 μg/ml) for 24 h. Cells were then injected into the footpad of syngenic mice. In some experiments, 20 or 100 μg of IL-1Ra (Anakinra) or 250 ng of recombinant murine IL-1β (or 250 ng of IL-6 or 50 ng of IL-12) was injected along with dying tumor cells into the footpad of mice. Alternatively, DC were cocultured with EG7 cells for 2 hours, followed by the purification of CD11c⁺ cells with anti-CD11c mAb coupled to magnetic beads (Miltenyi Biotec, Paris, France) and injection of these purified DC into the footpad. Five days later, gangliocytes from popliteal lymph nodes were harvested, seeded in 96 U-well plates (3.10⁵ cells per well), and restimulated with 1 mg/ml OVA protein. Restimulation with MCA205 or CT26 cells was performed using 3×10⁴ tumor cells killed by 5 min heating at 42° C. followed by 1 cycle of freezing/thawing in liquid nitrogen. For B16F10, restimulation was achieved using gp100 peptide as previously described (Overwijk et al., 1998). As a positive control of T cell priming, mice were injected with OVA protein (1 mg) (or gp100 for B16F10) and the association of CpG (10 μg) and Poly I:C (5 μg). For CT26 priming experiments, cell lysate plus LPS (250 ng) was used as a positive control. Supernatants were harvested 72 hours later, and IFNγ secretion was assessed by ELISA. Alternatively, cells were stimulated with PMA (10 ng/ml) and ionomycin (1 μg/ml) for 1 h, followed by the addition of brefeldin A (10 μg/ml for 6 h at 37° C.) (Apetoh et al., 2007), surface staining with Abs specific for CD3, CD4 or CD8, fixation and permeabilization (Cytofix/cytoperm kit) and then labeled with antibodies specific for IFNγ. Immunofluorescence was analyzed on a LSRII flow cytometer (BD Biosciences) with FACSDiva software.

For cross priming assays, 3.10⁵ X-Ray irradiated (10 Gy) TS/A OVA cells were injected into the footpad of C57BL/6 mice. Five days later, gangliocytes from popliteal lymph nodes were harvested, seeded in 96 U-well plates (3.10⁵ cells per well), and restimulated with 1 mg/ml OVA protein or 2 μg/ml SIINFEKL peptide. Supernatants were harvested 72 hours later and IFNγ secretion was assessed by ELISA. For priming assay with macrophages, 5×10⁶ peritoneal macrophages from Casp1^{-/-} were either untreated or treated with oxaliplatin (5 μg/ml) for 24 h. Cells were then injected into the footpad of mice. Five days later, gangliocytes from popliteal lymph nodes were harvested, seeded in 96 U-well plates (3.10⁵ cells per well), and restimulated with 1 mg/ml OVA protein. Supernatants were harvested 72 hours later and IFNγ secretion was assessed by ELISA

ATP Assays

1×10⁵ EG7 or CT26 cells were treated with oxaliplatin (5 μg/ml) or doxorubicin (20 μM), respectively, and intracellular and extracellular ATP were measured at 0, 4, 8, 12, 16, 20 and 24 hours. Briefly, supernatants were harvested and extracellular ATP was assessed using the luciferin-based ENLITEN ATP Assay (Promega, Madison, USA). 100 μl of luciferin-luciferase solution was added to the supernatants, and light emission was recorded. Addition of luciferin-luciferase solution caused an almost instantaneous burst of luminescence. Light emission was calibrated by using standard samples furnished by the manufacturer. Intracellular ATP was measured using a commercial ATP Assay kit (Calbiochem, Darmstadt, Germany). Cells were treated with a lysis solution containing 1% trichloroacetic acid for 1 min. The luciferin-luciferase solution was then added, and ATP measured. For ATP depletion, cells were first treated with either oxaliplatin or doxorubicin for 24 hours, and then incubated with antimycin A (2 μg/ml) and 2-deoxyglucose (30 mM) for 20 minutes, and ATP was subsequently measured as previously described (Holmsen et al., 1974). Plates were read in a Fluostar luminometer (BMG Labtech).

Chemotherapy and Radiotherapy of Tumors Established in Mice

Wild type or loss-of-function C57BL/6 mice were injected s.c. with 10⁶ EL4 cells into the right flank. Alternatively, wild-type BALB/c mice were injected with 5.10⁵ CT26 cells into the right flank. Mice were then randomly assigned to treatment groups of 4-6 mice each. The tumor surface was monitored using a caliper. When tumor size reached 70-90 mm² for EL4 (9-12 days post-injection) or 60-80 mm² for CT26 (8-10 days post-injection), mice received chemotherapy (Apetoh et al., 2007). Mice bearing EL4 tumors were injected with oxaliplatin (5 mg/kg i.p), while mice bearing CT26 tumors were injected with doxorubicin (50 μl, 4 mM i.t). In some experiments, CT26 tumor-bearing mice received two injections (on day 0 and day 2 after the commencement of chemotherapeutic treatment) of IL-1Ra (McIntyre et al., 1991) (100 μg), anti-IL-1 antibody (100 μg) or hamster serum as a control.

In vivo Priming Assay.

WT or Casp1^{-/-} mice were injected into the leg thigh with 10⁶ EG7 cells. When tumor size reached 70-90 mm², mice were treated with oxaliplatin (5 mg/kg body weight i.p). Five days later, gangliocytes from inguinal lymph nodes were harvested, and restimulated with 1 mg/ml OVA protein. Supernatants were harvested 72 hours later and IFNγ secretion was assessed by ELISA.

Anti-Cancer Vaccination

Anti cancer vaccination was assessed using a previously described setting (Casares et al., 2005; Obeid et al., 2007). CT26 cells were cultured with either PBS or 20 μM doxorubicin, and MCA205 with either PBS or 1 μM mitoxantrone for 24 h. All these treatments led to ˜50% of Annexin V⁺ DAPI⁺ cells, as assessed by FACS analysis at 24 hours. After careful washes, cells were resuspended in sterile PBS. CT26 cells were incubated in the presence or in the absence of antimycin A and deoxyglucose (2 μg/ml and 30 mM, respectively) for 20 min, or in the presence or in the absence of Oxi-ATP (100 mM). After 3 washes, 3×10⁶ dying CT26 cells (or 3×10⁵ MCA205 cells) were injected subcutaneously into the left flank of mice. In some experiments, 100 μg of IL-1Ra (Anakinra) was injected along with the dying cells. Seven days later, mice were rechallenged into the right flank with 5×10⁵ live CT26 cells (or, alternatively, 3.10⁴ live MCA205 cells). Tumor growth was then monitored weekly.

Immunofluorescence

EG7 cells were cultured with PBS or oxaliplatin for 24 hours, and then treated with either PBS, OxiATP (Lowe et al., 1982; Murgia et al., 1993) (100 mM), or antimycin A and deoxyglucose (2 μg/ml and 30 mM respectively for 20 minutes). Tumor cells were then carefully washed and cocultured with BMDC obtained from wild type or loss of function mice for 24 hours. While coculture supernatants were kept for ELISA assays, DC were harvested and washed, and seeded on polylysine slides for 30 min. DC were then fixed and permeabilized with BD Cytofix/cytoperm (Beckton Dickinson), following the manufacturer's instructions. Cells were then stained with a rabbit polyclonal caspase-1 p20 antibody (Santa Cruz Biotechnology, Inc, Santa Cruz, USA), CD11c Alexa Fluor 488 (Biolegend, San Diego, USA), and DAPI for immunofluorescence microscopy.

In vitro Stimulation of OT-1 Cells CD8⁺ T cells were purified from OT-1 spleens with a CD8⁺ T Cell Isolation Kit (Miltenyi Biotec, Paris, France). These responder cells (2×10⁴) were cocultured with bone marrow-derived DC from WT or Casp1 deficient mice (1×10⁴) loaded with live or oxaliplatin-treated EG7 cells (1×10⁴) in the presence of a control or anti-IL-1β antibody (10 μg/ml) round-bottom 96-well plates. Alternatively, OT-1 cells and BM-DC were incubated with OVA protein alone or with CpG (10 μg/ml) and Poly I:C (5 μg/ml). The supernatants were collected after 48 hours and assayed for IFNγ by ELISA.

In vitro Stimulation of CD8⁺ T-Cells

Double positive CD3⁺ and CD8⁺ cells were isolated from mouse spleen by cell-sorting on a MoFlo cytofluorometer (Dako Colorado Inc) and these cells were incubated in flat-bottom 96-well culture plates precoated with anti-CD3ε mAb (1 μg/ml) and anti-CD28 mAb (0.5 μg/ml) supplemented with various amounts of cytokines. The supernatants were collected after 48 hours and assayed for IFNγ by ELISA.

Statistical Analysis of Experimental Data

For the analysis of experimental data, comparison of continuous data was achieved by the Mann-Whitney U test, and comparison of categorical data by Chi-square or Fisher's exact test, as appropriate. The log rank test was used for the analysis of Kaplan Meier survival curves. Statistical calculations were performed with JMP 5.1 software (SAS Institute, Cary, N.C.). All p values were two-tailed. A p value<0.05 was considered statistically significant for all experiments.

Clinical Study Design

The inventors retrospectively constructed a patient database using data obtained from Institut Gustave Roussy and Centre René Huguenin (France). All patients provided written informed consent for enrollment in the study and approval was obtained from the local institutional review board “CCPPRB du Val de Marne”. Eligible patients had histologically confirmed, axillary node positive-sporadic breast cancer. Patients were selected to have been treated with primary surgery (+/− radiation therapy according to surgical procedure and local guidelines). All patients received an adjuvant anthracycline-based chemotherapy after surgery (FEC protocol). The performance of taxanes in adjuvant setting was not an exclusion criteria.

Patients with evidence of metastasis at the time of diagnosis or with incomplete surgical resection of the primary tumor were excluded from the study. Adjuvant endocrine therapy was recommended to all patients with hormone receptor positive tumors. Age at diagnosis, pathological tumor size, lymph node involvement, tumor grade, hormone receptors, endocrine treatments, occurrence of events and follow-up were extracted from medical files and recorded in the database (Table 1 below). The primary endpoint of the study was metastasis free survival, defined as time from diagnosis to the occurrence of metastasis. Data for patients who remained free of metastic event were censored at 10 years, the date of last visit according to French recommendation and because true recurrence after 10 years was exceptional (Brewster et al., 2008). After generation of the patient database and collection of genomic DNA samples, genotyping and statistical analyses were performed in a blinded fashion. A total of 230 patients fulfilled the inclusion criteria. Among those, 5 were excluded for node negative disease. Chi-square tests were used to compare the distribution of clinical characteristics across the two genotype groups. Survival rates were estimated using the Kaplan-Meier method. An univariate unstratified Cox model was then used. Significant data filtered by univariate unstratified Cox analysis were used to generated a multivariate Cox model. All analyses were carried out using SAS software, version 8.2 (SAS Institute Inc., Cary, N.C.).

TABLE 1
Breast cancer patient characteristics

| | P2X7 | P2X7 | |
|---|---|---|---|
| | (Glu496Glu) | (Glu496Ala) | |
| | (n = 144) | (n = 81) | p values |
| Mean Age (years) | 49 ± 11 | 50 ± 11 | NS |
| Lymph node involvement - n (%) | | | |
| 1-3 positive lymph nodes (pN1) | 87 (60) | 45 (54) | |
| 4-9 positive lymph nodes (pN2) | 45 (31) | 25 (31) | NS |
| ≧10 positive lymph nodes (pN3) | 12 (8) | 12 (15) | |
| Mean No. of positive lymph nodes | 4.8 ± 5.7 | 4.1 ± 4.2 | NS |
| Tumor size - n (%) | | | |
| 0-2 cm (pN1) | 38 (27) | 28 (35) | |
| 2-5 cm (pT2) | 89 (62.2) | 47 (58) | NS |
| >5 cm (pT3, pT4) | 16 (11) | 6 (7) | |
| Mean pathological tumor size (cm) | 2.8 ± 1.9 | 2.5 ± 1.7 | NS |
| Histologic grade of tumor - n (%) | | | |
| 3 Poorly differentiated | 60 (43) | 29 (39) | |
| 2 Moderatly differentiated | 66 (48) | 39 (45) | NS |
| 1 Well differentiated | 13 (9) | 7 (9) | |
| Hormone receptor status - n (%) | | | |
| Positive | 36 (25) | 24 (28) | |
| Negative | 104 (72) | 55 (65) | NS |
| Missing information | 5 (4) | 6 (7) | |
| Follow up (months) Mean ± SD | 71 ± 41 | 78 ± 42 | NS |

pT: pathological tumor size.
pN: pathological Nodal involvement.
Where applicable, data are presented as means ± standard deviation.

Genotyping of P2X7 Single Nucleotide Polymorphisms (SNPs)

DNA was isolated from frozen blood leukocytes from subjects. PCR primers (Applied Biosystems) were used to amplify a fragment containing the P2X7 Glu496Ala SNP (rs3751143). After PCR amplification, genotypes were assigned to each subject, by comparing the signals from the two fluorescent probes, FAM and VIC, and calculating the −log(FAM/VIC) ratio for each data point.

Results

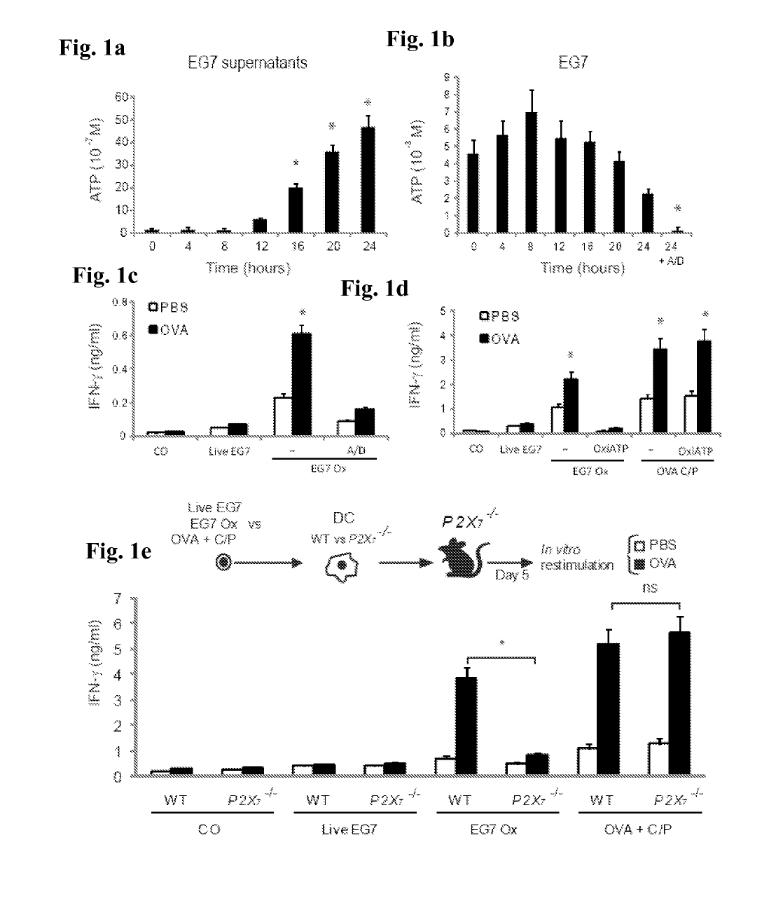

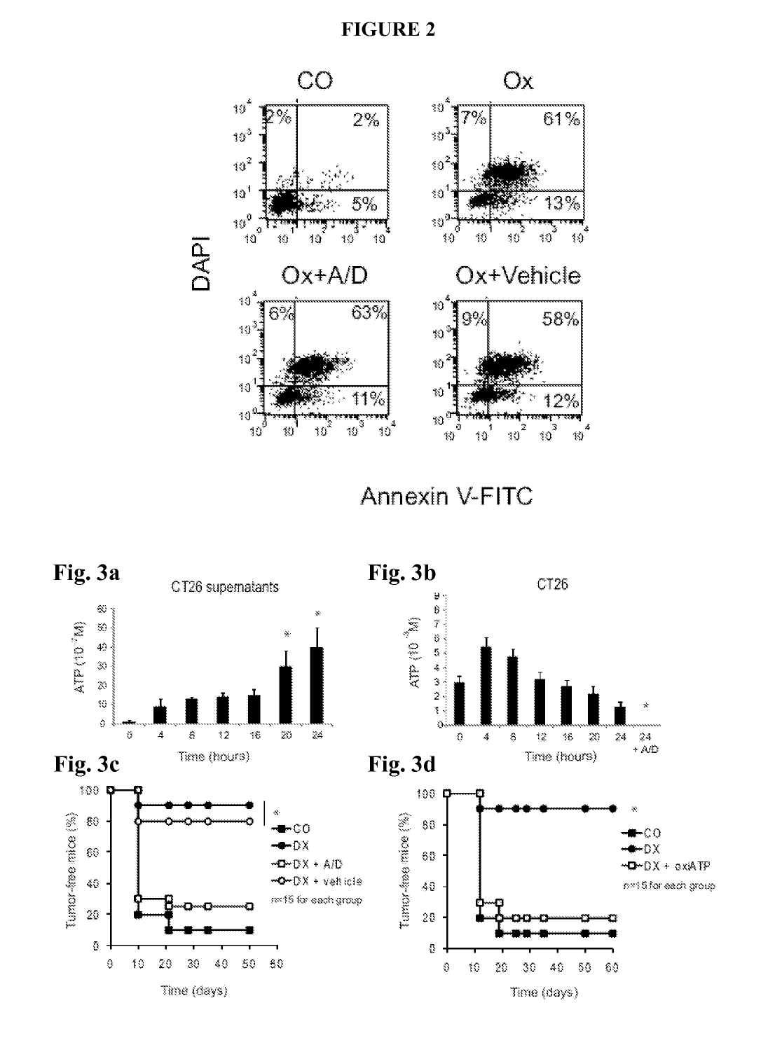

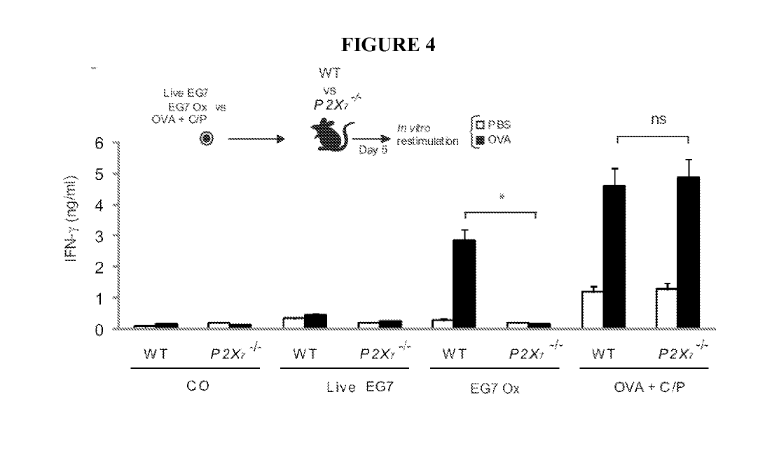

The inventors observed that oxaliplatin induced the release of ATP from multiple cell lines including EL4 thymomas and EG7 cells, which are ovalbumin (OVA)-transfected EL4 cells (FIG. 1a). Depletion of ATP by short-term treatment with deoxyglucose plus antimycin A (FIG. 1b), a treatment that did not induce necrosis (FIG. 2), abolished the immunogenicity of oxaliplatin-treated EG7 cells. After injection into the footpad, ATP-containing (but not ATP depleted), oxaliplatin-treated EG7 cells primed T cells from the popliteal lymph node to secrete IFNγ in response to restimulation with OVA (FIG. 1c). Blockade of purinergic receptors with the 2′,3′-dialdehyde derivative of ATP (“oxiATP”) abolished T cell priming by oxaliplatin-treated EG7 cells, yet did not affect T cell priming triggered by OVA protein plus adjuvant (FIG. 1d). Similar results were obtained for CT26 colon cancer cells, which released ATP in response to doxorubicin and which lost their potential to act as anti-cancer vaccine when ATP was depleted or purinergic receptors were blocked (FIG. 3). Oxaliplatin-treated EG7 cells failed to prime T cells for IFNγ production when they were inoculated into mice lacking the purinergic receptor P2X7, which has the highest affinity for ATP (FIG. 4). Bone marrow-derived DC (BM-DC) from WT (but not from P2X₇^{-/-}) mice primed T cells for IFNγ production when they were pre-incubated with dying EG7 cells and then inoculated into P2X₇^{-/-} mice (FIG. 1e), indicating that it is the P2X₇ receptor on DC (as opposed to other cell types) that senses ATP from dying tumor cells.

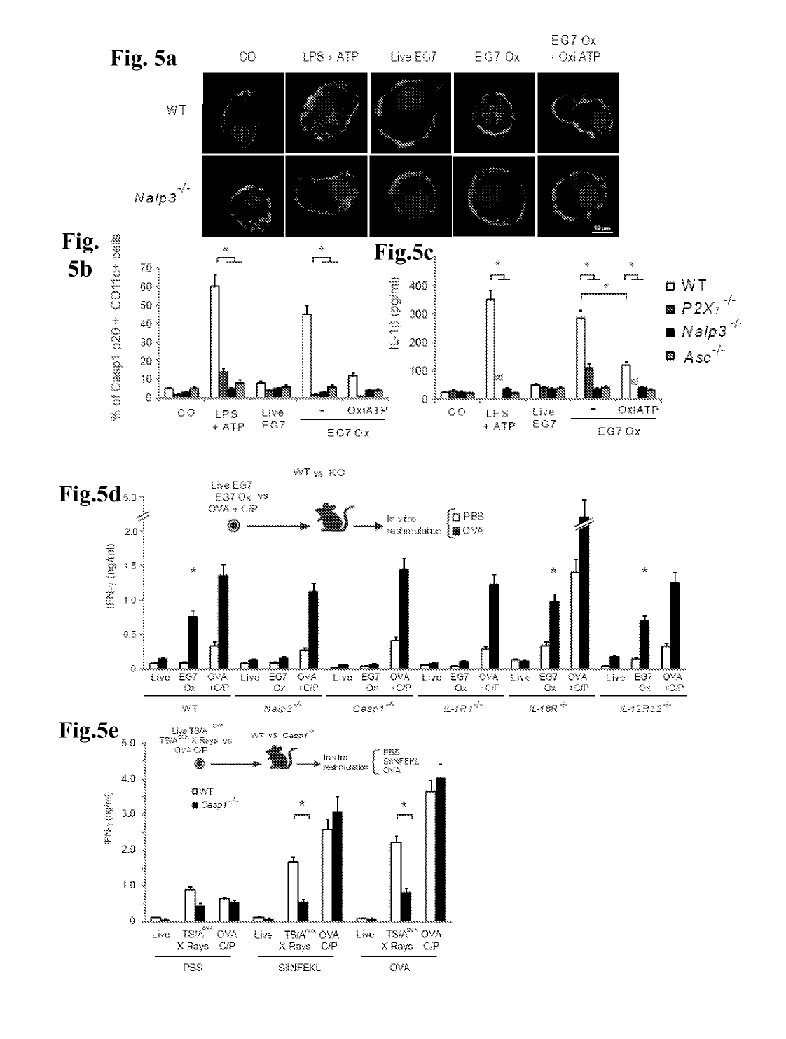

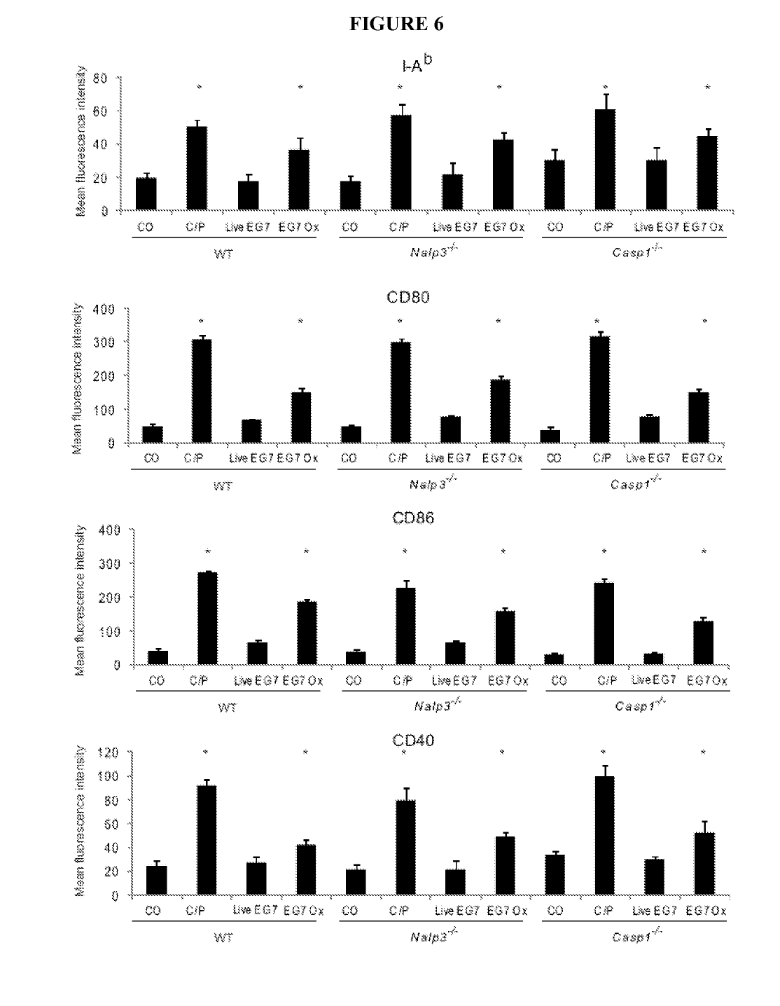

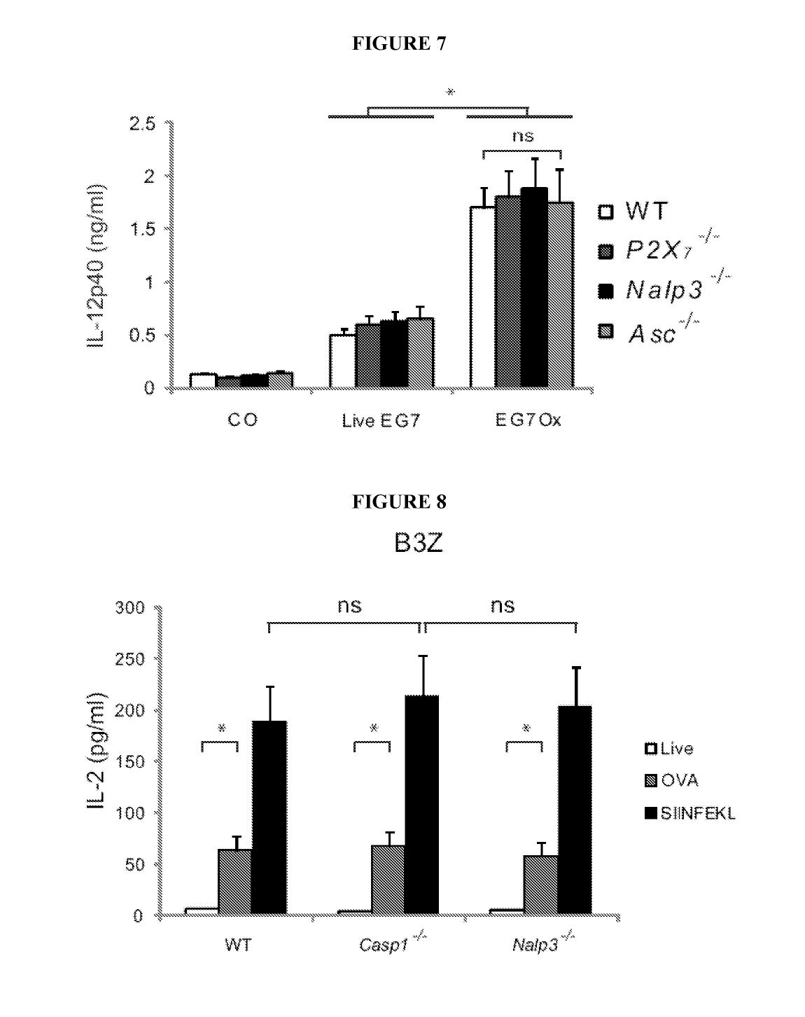

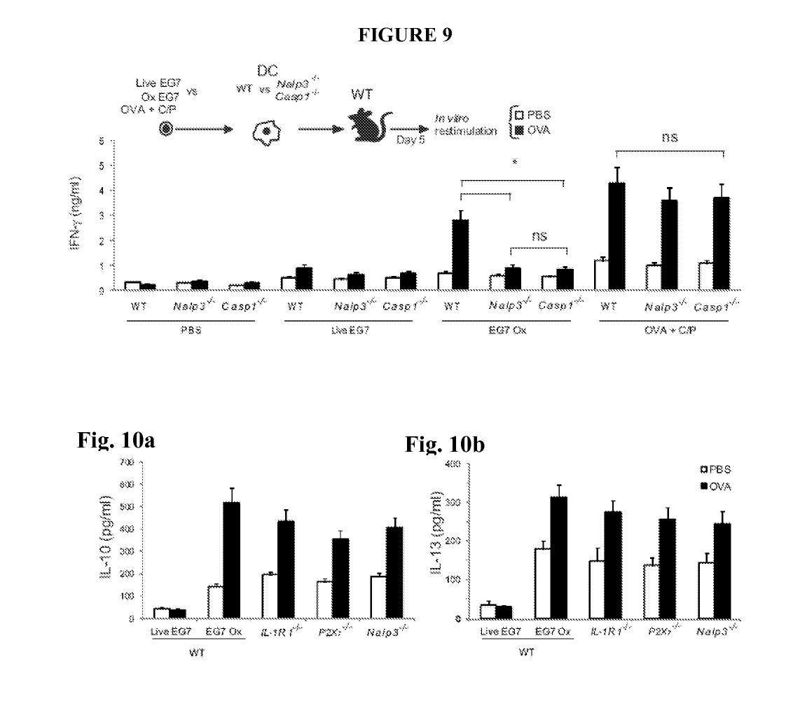

Via P2X₇ receptors, ATP can induce the NALP3-dependent proteolytical activation of caspase-1. Isolated WT BM-DC (but not P2X₇^{-/-}, Asc^{-/-} or Nalp3^{-/-} DC) incubated with dying EG7 cells activated caspase-1 (FIG. 5a,b). Caspase-1 activation within DC led to the secretion of IL-1β (FIG. 5c) but not IL-18 (which remained undetectable). The secretion of the IL-12 p40 subunit and the surface expression of MHC class II antigen, CD40, CD80 or CD86 by DC stimulated with dying tumor cells was independent of P2X₇, Nalp3 or Asc (FIG. 6, 7), indicating that failure to activate the inflammasome has a specific (rather than broad) effect on DC. Accordingly, Nalp3^{-/-} or Casp1^{-/-} DC loaded with dying EG7 cells presented the OVA-derived SIINFEKL peptide to T cell hybridoma cells as efficiently as WT DC (FIG. 8). In contrast to WT DC, Nalp3^{-/-} or Casp1^{-/-} DC pulsed with dying tumor cells failed to prime T cells for IFNγ production in vivo (FIG. 9). T cell priming for IFNγ production by syngeneic dying tumor cells was suppressed in Nalp3^{-/-}, Casp1^{-/-} or Il-1R1^{-/-} mice (but normal in Il-12Rβ2^{-/31} and IL-18R^{-/-} mice) (FIG. 5d). The absence of a functional P2X₇NALP3/Casp-1/IL-1R1 axis did not abolish T cell priming as such but rather caused a deviation towards a distinct cytokine pattern, in thus far that antigen-specific T cells normally produced IL-13 and IL-10 (FIG. 10) but not IFNγ (FIG. 5d).

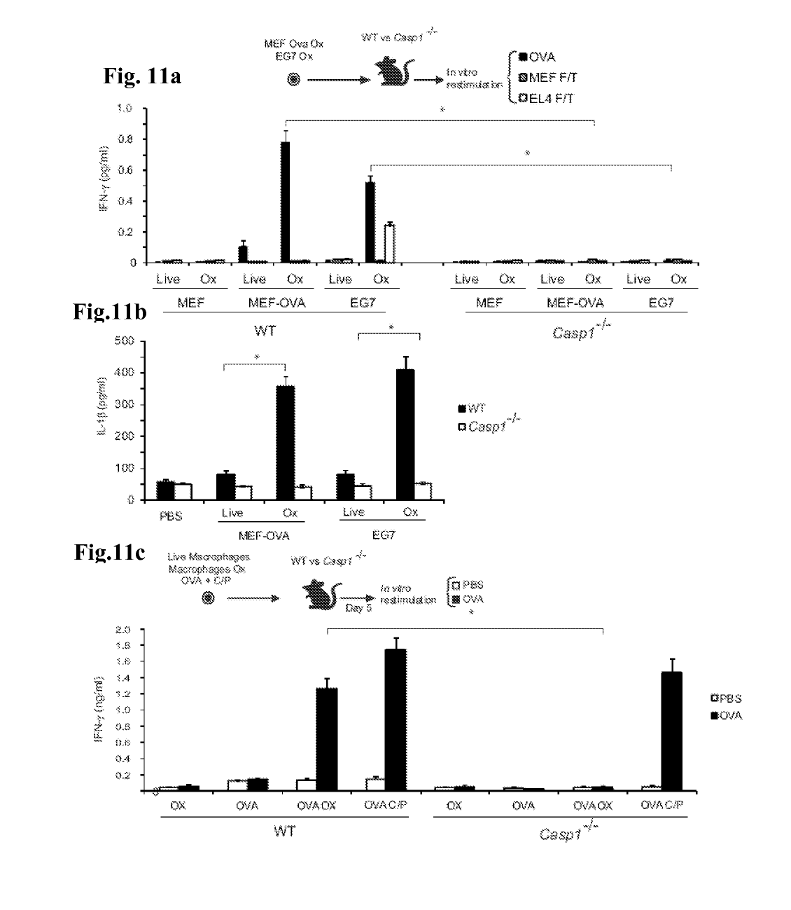

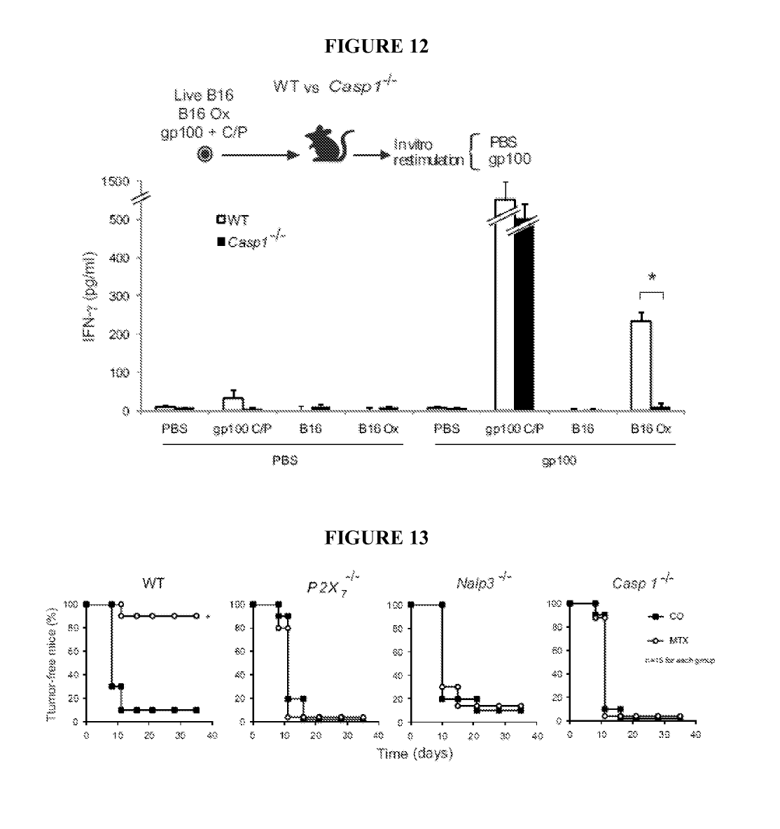

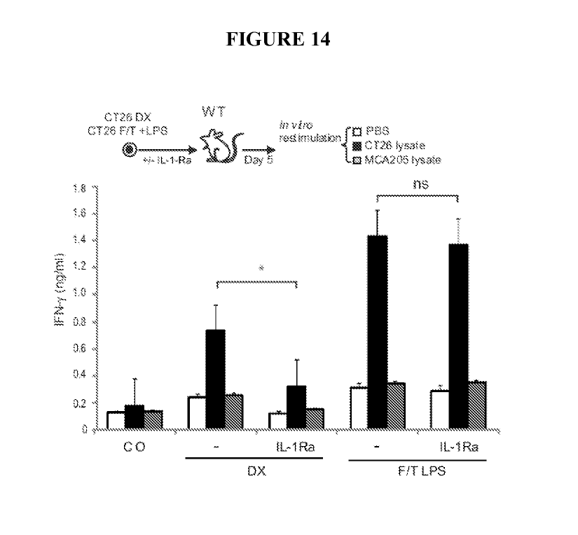

Cross-priming of MHC class I-restricted CD8⁺ T cells in an allogeneic (MHC-incompatible) system also depended on caspase-1. Dying OVA-expressing breast cancer cells (TS/A, H-2^{d}) failed to prime CTL specific for the OVA-derived, H-2^{b}-restricted SIINFEKL epitope when injected into Casp1^{-/-} mice (FIG. 5e). Similarly, dying mouse embryonic fibroblasts (MEF) expressing cell-associated OVA (fused to the transmembrane domain of K^{b} protein) primed T cells for IFNγ production when injected into WT but not Casp1^{-/-} mice (FIG. 11a,b). Casp-1 deficiency also compromised T cell priming by oxaliplatin-treated OVA-loaded peritoneal macrophages (FIG. 11c) and oxaliplatin-treated B16F10 melanoma cells (FIG. 12). Mitoxantrone-treated MCA205 fibrosarcoma cells elicited an immune response that prevented the growth of live tumor cells in WT mice but not in P2X₇^{-/-}, Nalp3^{-/-} or Casp1^{-/-} mice (FIG. 13). Dying CT26 cells primed T cells for IFNγ-production in response to autologous CT26 lysate (but not control lysates from a distinct tumor). This tumor-specific response was abolished by injection of recombinant IL-1 receptor antagonist (IL-1Ra) (FIG. 14). These results identify NALP3 inflammasome-dependent IL1β production as a critical element of CTL polarization/priming towards IFNγ production.

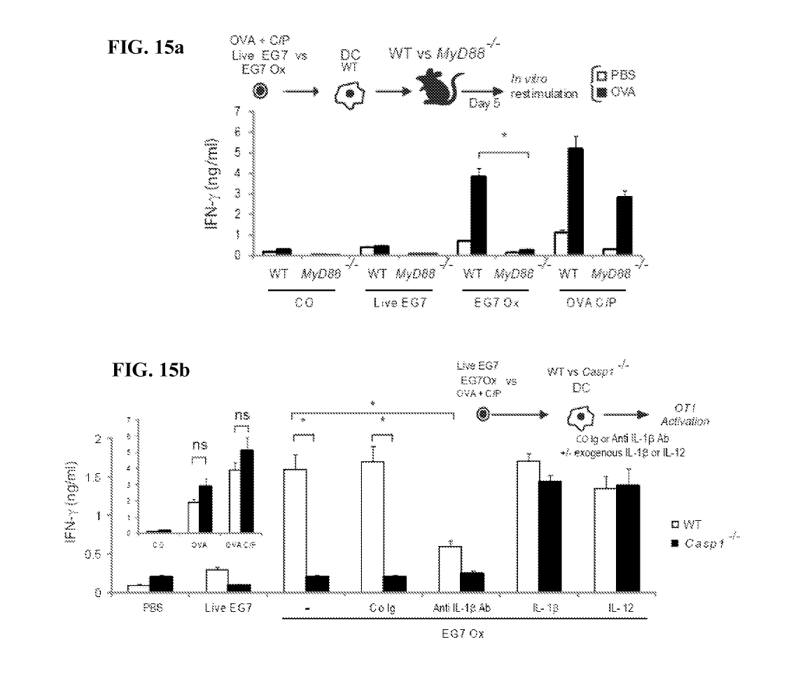

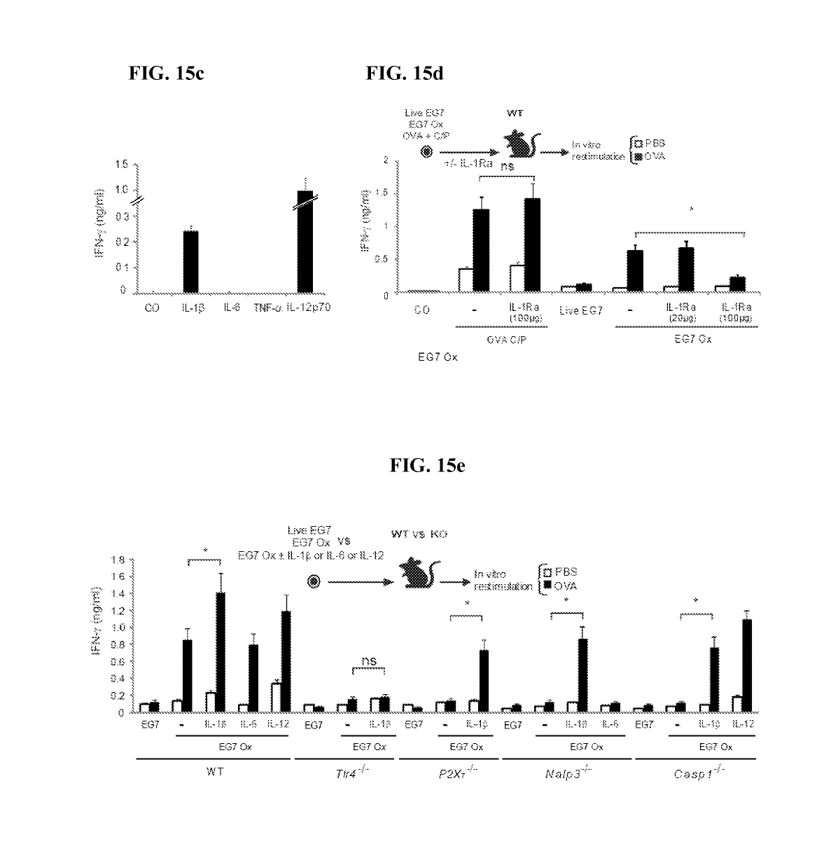

WT DC failed to activate T cells in hosts deficient for the adaptor molecule MyD88 that is indispensable for transducing the IL-1R-mediated signal in T cells (FIG. 15a), indicating that IL-1β signaling is critical for host cells (and not only at the level of the OVA-presenting DC). To directly assess the contribution of IL-1β to T cell priming, the inventors performed in vitro assays in which WT or Casp-1^{-/-} BM-DC were pulsed with dying tumor cells and then cocultured with na-ve T cells in the presence of IL-1Ra or IL-1β-neutralizing antibodies, followed by detection of secreted IFNγ. When H-2^{b}-expressing DC from Casp-1^{-/-} mice were pulsed with dying EG7 cells, they failed to prime naive T cells from OT-1 mice, which express a transgenic TCR that recognizes the OVA-derived SIINFEKL peptide presented by H-2^{b} class I molecules (FIG. 15b). In similar conditions, WT DC did stimulate OVA-specific OT-1 cells to produce IFNγ, unless IL-1β-specific antibodies were added to the system. Exogenous IL-1β or rIL-12 restored the priming capacity of Casp-1^{-/-} DC in vitro (FIG. 15b). Of note, WT and Casp-1^{-/-} DC promoted OT-1 activation with a similar efficacy, if they were pulsed with soluble OVA protein plus adjuvant instead of dying OVA-expressing cells (inset, FIG. 15b). To demonstrate a direct effect of IL-1β on CD8⁺ T cells, the inventors suboptimally stimulated CD3⁺CD8⁺ splenic T cells by cross-linking CD3 plus CD28, in the absence or presence of IL-1β. Naïve CTL failed to produce IFNγ, unless IL-113 (or IL-12 as a positive control, but not TNFα or IL-6 as negative controls) was added (FIG. 15c).

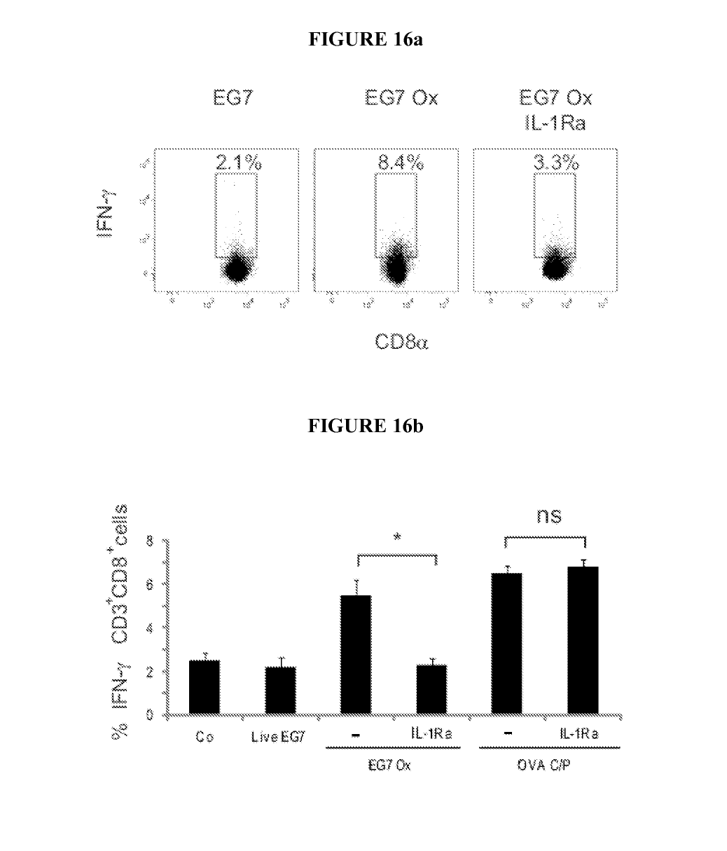

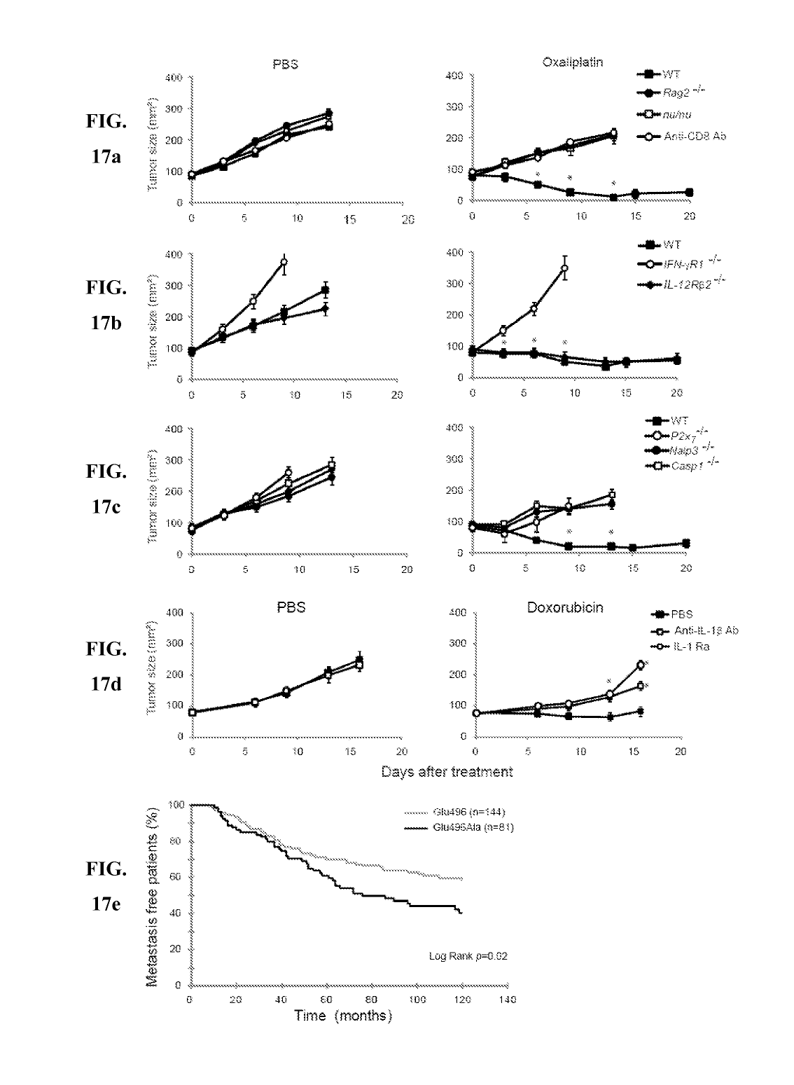

Oxaliplatin-treated EG7 cells failed to prime T cells in vivo, if they were co-injected into the footpad with IL-Ra. In these conditions, T cells from the draining lymph node failed to secrete IFNγ in vitro after restimulation with OVA. (FIG. 15d) and, more specifically, CD3⁺CD8⁺ T lymphocytes failed to stain positively for cytoplasmic IFNγ (FIG. 16). Conversely, local injection of recombinant IL-1β protein (or IL-12, but not IL-6), together with oxaliplatin-treated EG7 cells, restored deficient T cell priming in Nalp3^{-/-} or Casp1^{-/-} mice (FIG. 15e). Thus, IL-1β is the cytokine that determines the NALP3-dependent immunogenicity of cancer cell death.

Oxaliplatin was efficient in controlling EL4 tumor growth in immunocompetent WT mice. Oxaliplatin lost its therapeutic efficacy on tumors established in T and B cell-deficient rag-2^{-/-} mice, T cell-deficient nu/nu mice, mice that have been depleted from CD8⁺ T cells (FIG. 17a) and IfnγR1^{-/-} mice, yet remained efficient in WT and Il-12Rβ2^{31 /-} mice (FIG. 17b). Similarly, EL4 tumors implanted in P2X7^{-/-}, Nalp3^{-/-} or Casp1^{-/-} mice responded less efficiently to oxaliplatin than tumors growing in WT controls (FIG. 17c). IL-1Ra or anti-IL-1β also blunted the response of EL4 tumors to oxaliplatin (not shown) and CT26 tumors to anthracyclines in vivo (FIG. 17d). In all these models, as well as in patients with non-metastatic breast cancer who were treated with anthracyclines, deletion or loss-of-function alleles of TLR4 compromises the efficacy of chemotherapy, supporting the contribution of the immune system to the chemotherapeutic response. The inventors therefore sought whether a loss-function-allele that affects P2X₇ (Glu496Ala), reducing its affinity for ATP and hence ATP-induced IL-1β release, would accelerate therapeutic failure in anthracyclin-treated breast cancer patients (Table 1 above). The P2X₇ loss-of-function-allele enhanced the probability of metastatic relapse within ten years after diagnosis (52% in patients carrying the Glu496Ala allele versus 36% in patient with the normal allele; p=0.02 by Chi² analysis) and had a significant negative prognostic impact on metastasis-free survival (log-rank test; p=0.02) with a hazard ratio of 1.6 [95% confidence interval 1.1-2.4; p=0.02 in univariate Cox proportional hazard analysis) (FIG. 17e). These results lend support to the clinical relevance of the P2X₇/inflammasome pathway.

The present results identify a new signal that is required for cancer cell death to be immunogenic, namely the release of ATP. Extracellular ATP has been previously shown to mediate potent pro-inflammatory effects in severe tissue damage including myocardial infarction and hepatotoxic insult. The present data are compatible with a scenario in which ATP activates P2X₇ receptors on DC, thereby stimulating the sequential aggregation of the NALP3/ASC/Casp-1 inflammasome, the proteolytic maturation/activation of caspase-1, and the maturation/secretion of IL-1β. IL-1β then participates in the priming of IFNγ-producing tumor antigen-specific CD8⁺ T lymphocytes. Failure to execute any among the individual steps in this linear cascade may compromise the immune response against dying tumor cells and hence reduce the therapeutic efficacy of anti-cancer chemotherapies.

Example of Protocol to Assess the Sensitivity of a Subject to an Anticancer Treatment by Measuring the IL-1β Secretion Capacity of its Dendritic Cells in Presence of Dying Tumor Cells

MD-DC were obtained and cultured at the concentration of 2.10⁶/m1 on a 6-well plate in AIMV medium together with GM-CSF (800 IU/ml) and IFN-alpha (1000 IU/ml) for 2 days. HCT116 cells were treated with Oxaliplatin (10 ug/ml) for 16 hours. Dying HCT116 were then harvested and cocultivated with MDDC at a ratio of 1:1 in 96 well plate for 20 hours. As a positive control, DC were incubated with LPS (5 ng/ml) for 20 hours, and ATP (2 mM) at the end for 15 minutes. As a negative control, DC were incubated alone, or with living tumor cells. After 20 hours, supernatants were harvested and IL-1β was assessed in the different conditions using an ELISA assay.

### REFERENCES

Apetoh, L. et al. Toll-like receptor 4-dependent contribution of the immune system to anticancer chemotherapy and radiotherapy. Nat Med 13, 1050-9 (2007).
Akira, S., Uematsu, S. & Takeuchi, O. Pathogen recognition and innate immunity. Cell 124, 783-801 (2006).
Balkwill, F., Charles, K. A. & Mantovani, A. Smoldering and polarized inflammation in the initiation and promotion of malignant disease. Cancer Cell 7, 211-217 (2005).
Beutler, B. et al. Genetic analysis of host resistance: Toll-like receptor signaling and immunity at large. Annu Rev Immunol 24, 353-89 (2006).
Brewster, A. M., et al. Residual risk of breast cancer recurrence 5 years after adjuvant therapy. J Natl Cancer Inst 100, 1179-1183 (2008).
Carpentier A, et al. Phase 1 trial of a CpG oligodeoxynucleotide for patients with recurrent glioblastoma. Neuro Oncol. January; 8(1):60-6 (2006).
Casares, N., et al. Caspase-dependent immunogenicity of doxorubicin-induced tumor cell death. J Exp Med 202, 1691-1701 (2005).
Figdor, C. G., de Vries, I. J., Lesterhuis, W. J. & Melief, C. J. Dendritic cell immunotherapy: mapping the way. Nat Med 10, 475-80 (2004).
Fleischmann R, Stern R, Iqbal I. Anakinra: an inhibitor of IL-1 for the treatment of rheumatoid arthritis. Expert Opin Biol Ther. August; 4(8):1333-44 (2004).
Fritz, J. H., Ferrero, R. L., Philpott, D. J. & Girardin, S. E. Nod-like proteins in immunity, inflammation and disease. Nat Immunol 7, 1250-7 (2006).
Greten, F. R., et al. IKKbeta links inflammation and tumorigenesis in a mouse model of colitis-associated cancer. Cell 118, 285-296 (2004).
Hagemann, T., Balkwill, F. & Lawrence, T. Inflammation and cancer: a double-edged sword. Cancer Cell 12, 300-301 (2007).
Holmsen, H., Setkowsky, C. A. & Day, H. J. Effects of antimycin and 2-deoxyglucose on adenine nucleotides in human platelets. Role of metabolic adenosine triphosphate in primary aggregation, secondary aggregation and shape change of platetets. Biochem J 144, 385-396 (1974).
Huang, S., et al. Immune response in mice that lack the interferon-gamma receptor. Science 259, 1742-1745 (1993).
Huston J M, et al. Splenectomy protects against sepsis lethality and reduces serum HMGB 1 levels. J Immunol. Sep. 1; 181(5):3535-9 (2008).
Jursik, C. et al. A quantitative method for routine measurement of cell surface P2X7 receptor function in leucocyte subsets by two-colour time-resolved flow cytometry. J Immunol Methods. Aug. 31; 325(1-2):67-77 (2007).
Kanneganti, T. D., et al. Bacterial RNA and small antiviral compounds activate caspase-1 through cryopyrin/Nalp3. Nature 440, 233-236 (2006).
Karttunen, J., Sanderson, S. & Shastri, N. Detection of rare antigen-presenting cells by the lacZ T-cell activation assay suggests an expression cloning strategy for T-cell antigens. Proc Natl Acad Sci USA 89, 6020-6024 (1992).
Koebel, C. M. et al. Adaptive immunity maintains occult cancer in an equilibrium state. Nature 450, 903-7 (2007).
Kono, H. & Rock, K. L. How dying cells alert the immune system to danger. Nat Rev Immunol 8, 279-89 (2008).
Krelin, Y., et al. Interleukin-1beta-driven inflammation promotes the development and invasiveness of chemical carcinogen-induced tumors. Cancer Res 67, 1062-1071 (2007).
Li, P., et al. Mice deficient in IL-1 beta-converting enzyme are defective in production of mature IL-1 beta and resistant to endotoxic shock. Cell 80, 401-411 (1995).
Lowe, P. N. & Beechey, R. B. Interactions between the mitochondrial adenosinetriphosphatase and periodate-oxidized adenosine 5′-triphosphate, an affinity label for adenosine 5′-triphosphate binding sites. Biochemistry 21, 4073-4082 (1982).
Lutz, M. B., et al. An advanced culture method for generating large quantities of highly pure dendritic cells from mouse bone marrow. J Immunol Methods 223, 77-92 (1999).
Martinon, F., Burns, K. & Tschopp, J. The inflammasome: a molecular platform triggering activation of inflammatory caspases and processing of proIL-beta. Mol Cell 10, 417-26 (2002).
McIntyre, K. W., et al. Inhibition of interleukin 1 (IL-1) binding and bioactivity in vitro and modulation of acute inflammation in vivo by IL-1 receptor antagonist and anti-IL-1 receptor monoclonal antibody. J Exp Med 173, 931-939 (1991).
Murgia, M., Hanau, S., Pizzo, P., Rippa, M. & Di Virgilio, F. Oxidized ATP. An irreversible inhibitor of the macrophage purinergic P2Z receptor. J Biol Chem 268, 8199-8203 (1993).
Muruve, D. A. et al. The inflammasome recognizes cytosolic microbial and host DNA and triggers an innate immune response. Nature 452, 103-7 (2008).
Naugler, W. E., et al. Gender disparity in liver cancer due to sex differences in MyD88-dependent IL-6 production. Science 317, 121-124 (2007).
Obeid, M. et al. Calreticulin exposure dictates the immunogenicity of cancer cell death. Nat Med 13, 54-61 (2007).
Ogura, Y., Sutterwala, F. S. & Flavell, R. A. The inflammasome: first line of the immune response to cell stress. Cell 126, 659-62 (2006).
Overwijk, W. W., et al. gp100/pmel 17 is a murine tumor rejection antigen: induction of “self”-reactive, tumoricidal T cells using high-affinity, altered peptide ligand. J Exp Med 188, 277-286 (1998).
Sluyter et al. Extracellular ATP increases cation fluxes in Human erythrocytes by activation of the P2X7 receptor. J. Biol. Chem., 279, 44749-44755 (2004).
Solle, M., et al. Altered cytokine production in mice lacking P2X(7) receptors. J Biol Chem 276, 125-132 (2001).
Srinivasula, S. M., et al. The PYRIN-CARD protein ASC is an activating adaptor for caspase-1. J Biol Chem 277, 21119-21122 (2002).
Steinman, R. M. & Banchereau, J. Taking dendritic cells into medicine. Nature 449, 419-26 (2007).
Ting, J. P., Willingham, S. B. & Bergstralh, D. T. NLRs at the intersection of cell death and immunity. Nat Rev Immunol 8, 372-9 (2008).
Wu, C., et al. IL-12 receptor beta 2 (IL-12R beta 2)-deficient mice are defective in IL-12-mediated signaling despite the presence of high affinity IL-12 binding sites. J Immunol 165, 6221-6228 (2000).
Zitvogel, L., Apetoh, L., Ghiringhelli, F. & Kroemer, G. Immunological aspects of cancer chemotherapy. Nat Rev Immunol 8, 59-73 (2008).

1-36. (canceled)

37. An in vitro method of assessing the sensitivity of a subject to a chemotherapeutic or radiotherapeutic treatment of cancer, wherein the method comprises determining the functional status of the P2X₇-elicited NALP3 inflammasome pathway, a non-functional pathway being indicative of a resistance to said treatment.

38. The method according to claim 37, wherein the functional status of the P2X₇-elicited NALP3 inflammasome pathway is assessed by the detection of a loss-of-function mutation in a gene involved in said pathway, the presence of said mutation being indicative of a non functional P2X₇-elicited NALP3 inflammasome pathway.

39. The method according to claim 38, wherein the mutation is a SNP selected from the group consisting of rs28360457, rs1653624, rs3751143, rs2230911 and rs501192.

40. The method according to claim 39, wherein the SNP is rs3751143.

41. The method according to claim 37, wherein the functional status of the P2X₇-elicited NALP3 inflammasome pathway is assessed by:
a) the detection of a mutated polypeptide sequence or an impaired expression of a protein involved in said pathway, a mutated sequence or an impaired expression being indicative of a non functional P2X₇-elicited NALP3 inflammasome pathway;
b) comparing the IL-1β level in a blood sample of the subject before and after a chemotherapeutic or radiotherapeutic treatment, a significant increase of said level after said treatment being indicative of a functional P2X₇-elicited NALP3 inflammasome pathway;
c) measuring the capacity of the dendritic cells of the subject to secrete IL-1β in presence of dying tumor cells, wherein a reduced capacity compared to a standard level is correlated to a non functional P2X₇-elicited NALP3 inflammasome pathway;
d) measuring the capacity of the dendritic cells of the subject to secrete IL-1β in presence of HMGB 1 and ATP, wherein a reduced capacity compared to a standard level is correlated to a non-functional P2X₇-elicited NALP3 inflammasome pathway; or
e) measuring the capacity of the monocytes of the subject to secrete IL-1β in presence of lipopolysaccharide and ATP, wherein a reduced capacity compared to a standard level is correlated to a non-functional P2X₇-elicited NALP3 inflammasome pathway.

42. The method according to claim 37, wherein the functional status of the P2X₇-elicited NALP3 inflammasome pathway is assessed by the functional analysis of components of said pathway, a loss of function of at least one component of said pathway being indicative of a non functional P2X₇-elicited NALP3 inflammasome pathway.

43. The method according to claim 42, wherein the functional status of the P2X₇-elicited NALP3 inflammasome pathway is assessed by:
a) functional analysis of the P2X₇ receptor;
b) functional analysis of IL-1β; or e) functional analysis of caspase-1.

44. The method according to claim 37, wherein the chemotherapeutic treatment of cancer is selected from the group consisting of anthracyclines, oxaliplatin and cisplatin.

45. The method according to claim 37, wherein the radiotherapeutic treatment is X-rays or gamma-rays.

46. The method according to claim 37, wherein the cancer is selected from the group consisting of breast, colon, ovarian, stomach, sarcoma, endometrium, bladder, cervix, prostate, rectum, lung, ORL cancer, paediatric tumors, neuroblastoma, glioblastoma multiforme, lymphoma, leukemia, myeloma, seminoma, Hodgkin and malignant hemopathies.

47. An in vitro method for screening a compound useful for increasing or restoring the sensitivity to a chemotherapeutic or radiotherapeutic treatment of cancer in a subject having a loss of function in the P2X₇-elicited. NALP3 inflammasome pathway, wherein the method comprises determining the ability of a test compound to induce or increase IL-10 secretion by dendritic cells in presence of dying tumor cells in said subject.

48. An in vitro method for screening a compound useful for treating a cancer in a subject having a loss of function in the P2X₇-elicited NALP3 inflammasome pathway, wherein the method comprises determining the ability of a test compound to induce or increase IL-1β secretion by dendritic cells in presence of dying tumor cells in said subject.

49. A method for screening a compound useful for increasing or restoring the sensitivity to a chemotherapeutic or radiotherapeutic treatment of cancer in a subject having a non-functional P2X₇-elicited NALP3 inflammasome pathway, wherein the method comprises (i) administering a test compound in combination with a chemotherapeutic or radiotherapeutic treatment of cancer to a non-human transgenic animal with non-functional P2X₇-elicited NALP3 inflammasome pathway and inoculated with a tumor, and (ii) assessing the sensitivity of said animal to said treatment.

50. An in vitro method for determining the likelihood of a metastatic relapse in a subject, which method comprises determining the functional status of the P2X₇-elicited NALP3 inflammasome pathway in said subject, a non functional pathway being indicative of an increaseD likelihood of a metastatic relapse.

51. An in vitro method for selecting the proper chemotherapeutic or radiotherapeutic treatment for a subject in need thereof, wherein the method comprises determining the functional status of the P2X₇-elicited NALP3 inflammasome pathway, a non functional pathway being considered as a contraindication for an anticancer treatment inducing immunogenic tumor cell death.

52. A pharmaceutical composition comprising a chemotherapeutic agent and a compound which is able to compensate a loss of function in the P2X₇-elicited NALP3 inflammasome pathway.

53. The pharmaceutical composition according to claim 52, wherein the chemotherapeutic agent is selected from the group consisting of anthracyclines, oxaliplatin and cisplatin.

54. The pharmaceutical composition according to claim 52, wherein the compound which is able to compensate a loss of function in the P2X₇-elicited NALP3 inflammasome pathway, is selected from the group consisting of IL-1β; IL-12; CD1d agonists, IL-15, IL-2 or IFNα; cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-10 monoclonal antibodies, anti-IL-13 monoclonal antibodies, anti-PDL-1 monoclonal antibodies and IL-33.

55. The pharmaceutical composition according to claim 54, wherein the compound which is able to compensate a loss of function in the P2X₇-elicited NALP3 inflammasome pathway, is recombinant IL-1β.

56. A method for increasing the efficacy of a chemotherapeutic or radiotherapeutic treatment in a subject suffering from a cancer and having a non-functional P2X₇-elicited NALP3 inflammasome pathway, wherein the method comprises administering a chemotherapeutic or radiotherapeutic treatment in combination with a therapeutically effective amount of compound which is able to compensate a loss of function in the P2X₇-elicited NALP3 inflammasome pathway.

57. A method for treating a subject suffering from a cancer and having a loss of function in the P2X₇-elicited NALP3 inflammasome pathway, wherein the method comprises administering a chemotherapeutic or radiotherapeutic treatment in combination with a compound which is able to compensate a loss of function in the P2X₇-elicited NALP3 inflammasome pathway.

58. The method according to claim 57, wherein said compound is selected from the group consisting of IL-1β; IL-12; CD1d agonists, IL-15, IL-2 or IFNα; cathelicidin-derived peptide LL37, polymyxin B, STAT3 inhibitors, anti-CTLA4 antibodies, anti-PD-1 antibodies, TGFb inhibitory peptides, IL-10 inhibitory peptides, anti-IL-13 monoclonal antibodies and IL-33.

59. The method according to claim 58, wherein said compound is recombinant IL-1β.

60. The method according to claim 57, comprising administering a treatment selected from the group consisting of anthracyclines, oxaliplatin, cisplatin and X-rays, in combination with recombinant IL-1β.


SEQUENCE LISTING (5 sequences)

SEQ ID NO: 1 - DNA, 601 bp, Homo sapiens
  misc_feature (301)..(301): SNP rs3751143 (K = G/T)
cagtaaggactcttgaagccactgtactgcccttcactctttggaaactctttccggatcctccagcggcagcagctgggcaggatggcaaagtcagccatgtcctgggagccgaagcgccaggtggcgtagcacctgtaggcacagtgccgcagccggctgttggtggaatccacatccagcgccagcaagggctcctggtagagcaggaggaactgcaggacgtgtctggacaggaccagcttcctgaacagctctgaggtggtgatgcaggcccccggctttttccggcagcacagckcctccaggcacctgtggctctcagggagttgagatgggaggcagcttccacactggcaccagacggggctatccctggatctaggagtcgcctcctttctaagcagctgtatctcctctggttgtccaggaatcgggggtgtgtcatggagggccaggggcagcctggacaaatctgtgaagtccatcgcaggtctctgaaaaagcaagtttccaaacctattagccatgacaagccctcaggttctaggttctttaattttattaacttataaagttacatttaatattacatgactta

SEQ ID NO: 2 - DNA, 101 bp, Homo sapiens
  misc_feature (51)..(51): SNP rs28360457 (R = A/G)
ttcttcctacagatacgccaagtactacaaggaaaacaatgttgagaaacrgactctgataaaagtcttcgggatccgttttgacatcctggtttttggca

SEQ ID NO: 3 - DNA, 694 bp, Homo sapiens
  misc_feature (201)..(201): SNP rs1653624 (W = A/T)
aaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccawcctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgct

SEQ ID NO: 4 - DNA, 601 bp, Homo sapiens
  misc_feature (301)..(301): SNP rs2230911 (S = C/G)
gcatctatccaagtcacagcatgaggctccgctccctgatagaaccaacaattgcacgttgaagcaaaagagcgttgctctgaatttcacctgagtaaactctcccactctgtttttagggaggaaaatttgacattatccagctggttgtgtacatcggctcaaccctctcctacttcggtctggtaagagattctcttttccatgctttaggaaaatggtttggagaaggaagtgactaacgcagcgcttgtctgcattctccccaggccgctgtgttcatcgacttcctcatcgacasttactccagtaactgctgtcgctcccatatttatccctggtgcaagtgctgtcagccctgtgtggtcaacgaatactactacaggaagaagtgcgagtccattgtggagccaaagccggtgaggccgctgtgttcacaggacaccaagacatggagagattccatgaaatcactcagaaatgcacgaaaattaggcccaaatcacaggcttcatcctgtagtggatacgtcgctgggttctaccccgatcaaccaactctcagataaattttttggtcttagagaagaatggaaacaaaa

SEQ ID NO: 5 - DNA, 801 bp, Homo sapiens
  misc_feature (401)..(401): SNP rs501192 (R = A/G)
ccttcacctttcttgaaactaagactaaatttttttttggcaccctgttgtttgtcacggaattccttaagaaactccttaagtatttatttattgactttccaaggtaccatgatagttaattacatagtcgaaaatcagtgatatggtgtctttcaggacatgactacagagctggaggcatttgcacaccgcccagagcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcctgccgtggtggtgagtrctgatgcctttgagaacaccagacaatcaaatactatcctactctgtatgcatccaagaatttttggtttcttttggtttgtcttttaatgtcaaagaacacatactgtcagtattcaagaatgcagcttctgtgaattgaatggctggttggtagatatttattccatatttaagcttgagattttgatgtttggaaatttttacaatttgtttgatggttagaaaagataaacattgttgaaaagattgtaactgaaatgcatgtgtctatttgcatgtgaattaatgatagagcaaacacattaataaatatcattgaacagaggctaggaggaaaacatcttctcagccattggtagtagtaacaaatcaaactgtgtctttccagacagccctgg